(19) United States
(12) Patent Application Publication
(10) Pub. No.: US 2025/0189507 A1
(43) Pub. Date: Jun. 12, 2025

(54) SYSTEM FOR ROBOTIC CHARACTERIZATION OF IMPACTED SITES

(71) Applicants: Chevron U.S.A. Inc., San Ramon, CA (US); Carnegie Mellon University, Pittsburgh, PA (US)

(72) Inventors: Thomas Peter Hoelen, Berkeley, CA (US); Natasha Sihota, Danville, CA (US); Aaron M. Johnson, Pittsburgh, PA (US); Gregory V. Lowry, Pittsburgh, PA (US)

(21) Appl. No.: 18/847,016

(22) PCT Filed: Mar. 15, 2023
(86) PCT No.: PCT/US2023/015293

§ 371 Date: Sep. 13, 2024

Related U.S. Application Data
(60) Provisional application No. 63/320,177, filed on Mar. 15, 2022.

(51) Int. Cl. G01N 33/24; G01N 1/08; G05D 1/622; G05D 1/646; G05D 105/80; G05D 109/10

(52) U.S. Cl. CPC G01N 33/24; G01N 1/08; G05D 1/622; G05D 1/646; G05D 2105/80; G05D 2109/10

(57) ABSTRACT

A mobile testing and evaluation vehicle can include a body and a mobility feature disposed on the body, where the mobility feature is configured to move the body with respect to a zone of interest. The mobile testing and evaluation vehicle can also include a sensor device configured to obtain a measurement of a parameter associated with a sample in the zone of interest. The mobile testing and evaluation vehicle can further include a controller that is configured to receive the measurement, evaluate the sample based on the measurement, and evaluate the zone of interest based on evaluating the sample.

### CROSS-REFERENCE TO RELATED APPLICATIONS

This application claims priority to U.S. Provisional Patent Application Ser. No. 63/320,177 titled “System for Robotic Characterization of Impacted Sites” and filed on Mar. 15, 2022, the entire contents of which are hereby incorporated herein by reference.

### TECHNICAL FIELD

The present application is related to impacted sites and, more particularly, to systems for robotic characterization of impacted sites.

### BACKGROUND

Soils and other sites impacted with compounds of potential concern (COPC) need to be delineated at times to understand the extent of the impact, evaluate the potential for such compounds to reach receptors, or decide on site remediation strategies (e.g., following EPA RCRA site assessment). Examples of a COPC can include, but are not limited to crude oil, salt, certain metals, radioactive elements, and asbestos fibers. Site sampling in the current art is typically performed by a manual crew, which may raise human health and safety concerns, can be difficult, costly, and time consuming to implement, and can have the potential to lead to human errors. For example, after an initial round of exploratory sampling and subsequent analysis, several rounds of follow-up sampling are generally performed to identify ‘hot spots’ (i.e., areas with the highest concentrations of COPCs) and reduce uncertainty regarding distribution of COPCs with respect to cleanup goals.

High sampling density is generally considered more effective in reducing uncertainty related to the extent of impacts of COPCs than analytical quality because of highly heterogeneous distributions of COPCs at impacted sites. Sample analysis, which is performed in a laboratory (e.g., remote, on site) after the sample is acquired, is time consuming and expensive. Practical limitations related to keeping field crews mobilized while waiting for analytical results can limit the opportunity to collect follow-up samples.

Sampling and analysis with a human work crew is of particular concern for remote sites with terrain that is challenging and potentially dangerous to traverse and sample. For example, sites with dense vegetation, hills or slopes, unstable surfaces, marshes, areas that are prone to flooding, sites that could release flammable or toxic gases, and sites with animals or civil unrest can all pose hazards to human sampling crews. Manual sample collection and on-site analysis at such sites can be particularly expensive and time consuming, typically resulting in low sampling density and relatively poor delineation of possible environmental impacts. Analysis of soil samples for targeted follow-up sampling to refine delineation boundaries and identify hot-spots is also time consuming, leading to either increased costs for mobilized sampling personnel or to suboptimal selection of follow-up sampling locations. This can lead to poor risk evaluation, incorrect cost estimates, selection of inappropriate remediation technologies, and/or unnecessary remediation (e.g., excessive excavation). Also, manual sampling and data recording in the field can lead to cross contamination and errors in data documentation.

In some cases, portable analytical devices are used in the current art. However, the use of portable analytical devices (e.g., wet chemistry Total Petroleum Hydrocarbon (TPH) test kits, portable infrared (IR) devices, portable X-Ray Fluorescence (XRF) devices) can be time consuming and prone to human error, and portable analytical devices do not eliminate or sufficiently reduce the need for crews to collect and manually handle samples. The use of such devices by field crews may be cumbersome because of their size and/or weight. In other cases, the use of such devices is not possible when sites have the potential presence of flammable vapors because the devices are typically not intrinsically safe (e.g., explosion proof). In addition, site heterogeneity and specific requirements for certain analytical techniques require procedures to pre-treat samples prior to analysis. These procedures may include complex homogenizing, sample drying, and/or extraction with solvents, all of which can be labor intensive, time consuming, and ripe for opportunities for cross contamination, thereby causing further delay in the generation of useful data.

Robotic platforms are commonly used in agriculture, but these robotic platforms are typically used to perform routine tasks in highly homogenous and cultivated terrains. Similarly, robots for mining can drill cores and collect subsurface samples, but do not have the ability to characterize the samples on-board. These platforms also tend to be very large and heavy, which make them expensive and difficult to operate and/or transport.

### SUMMARY

In general, in one aspect, the disclosure relates to a mobile testing and evaluation vehicle. The mobile testing and evaluation vehicle can include a body and a mobility feature disposed on the body, where the mobility feature is configured to move the body with respect to a zone of interest. The mobile testing and evaluation vehicle can also include a sensor device configured to obtain a measurement of a parameter associated with a sample in the zone of interest. The mobile testing and evaluation vehicle can further include a controller that is configured to receive the measurement, evaluate the sample based on the measurement, and evaluate the zone of interest based on evaluating the sample.

In another aspect, the disclosure relates to a method for assessing a site with compounds of potential concern. The method can include moving, by a mobile testing and evaluation vehicle, to a plurality of locations in a zone of interest. The method can also include obtaining, by a mobile testing and evaluation vehicle, a plurality of measurements of a parameter associated with a plurality of samples in the zone of interest. The method can further include evaluating, by the mobile testing and evaluation vehicle, the plurality of samples based on the plurality of measurements. The method can also include evaluating, by the mobile testing and evaluation vehicle, the zone of interest based on evaluating the plurality of samples.

These and other aspects, objects, features, and embodiments will be apparent from the following description and the appended claims.

### BRIEF DESCRIPTION OF THE DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, reference numerals designate like or corresponding, but not necessarily identical, elements.

FIG. 1 shows a system for robotically characterizing impacted sites according to certain example embodiments.

FIG. 2 shows a system diagram of a mobile testing and evaluation vehicle of FIG. 1 according to certain example embodiments.

FIG. 3 shows a system diagram of a modular version of the mobile testing and evaluation vehicle of FIGS. 1 and 2 according to certain example embodiments.

FIG. 4 shows a system diagram of a controller of the mobile testing and evaluation vehicle of FIG. 2 according to certain example embodiments.

FIG. 5 shows a computing device in accordance with certain example embodiments.

FIG. 6 shows a flowchart of a method for assessing a site with compounds of potential concern according to certain example embodiments.

FIG. 7 shows a subsystem that includes an example of a mobile testing and evaluation vehicle according to certain example embodiments.

FIG. 8 shows a subsystem that includes another example of a mobile testing and evaluation vehicle according to certain example embodiments.

FIG. 9 shows a subsystem that includes yet another example of a mobile testing and evaluation vehicle according to certain example embodiments.

FIG. 10 shows a subsystem that includes still another example of a mobile testing and evaluation vehicle according to certain example embodiments.

FIG. 11 shows a subsystem that includes yet another example of a mobile testing and evaluation vehicle according to certain example embodiments.

FIGS. 12A and 12B show a subsystem that includes an example of a mobile testing and evaluation vehicle identifying objects in a zone of interest according to certain example embodiments.

FIG. 13 shows diagram of the subsystem of FIGS. 12A and 12B with a path generated by the example mobile testing and evaluation vehicle based on identifying the objects according to certain example embodiments.

FIGS. 14A and 14B show a subsystem that includes an example mobile testing and evaluation vehicle generating a path based on identifying an object in a zone of interest according to certain example embodiments.

FIGS. 15A and 15B show a subsystem that includes an example mobile testing and evaluation vehicle modifying a path based on evaluating samples in a zone of interest according to certain example embodiments.

### DESCRIPTION OF THE INVENTION

The example embodiments discussed herein are directed to systems and methods for robotic characterization of impacted sites. Example mobile testing and evaluation vehicles discussed herein can be used on land, in water, and/or in some other medium. Industries for which example mobile testing and evaluation vehicles can be used can include, but are not limited to, oil and gas (e.g., exploration, production, refining), chemical production and/or manufacturing, aquaculture, oceanography, and electric power (e.g., wind generation).

Example mobile testing and evaluation vehicles can be used to travel along and/or assess any of a number of structures, including but not limited to spars, tanks, semisubmersibles, tension leg platforms (TLPs), Floating Production Storage and Offloading (FPSOs), Floating Storage and Offloading (FSOs), and ships of any kind (e.g., tankers, barges). When example mobile testing and evaluation vehicles are used on land, examples of terrains (e.g., a surface (discussed below)) along which the mobile testing and evaluation vehicles can travel can include gravel, asphalt, grass, concrete, tile, sand, and larger rocks.

Example mobile testing and evaluation vehicles described herein can operate (e.g., collect samples, obtain measurements, generate and/or revise a path of travel, evaluate a zone of interest) autonomously, without being directed by a user. Example unmanned mobile testing and evaluation vehicles can be rated for use in sites that can be categorized as hazardous environments, explosive environments, underwater environments, humid environments, extremely hot and/or cold environments, dusty environments, windy environments, and/or other types of environments that may require specialized configurations in order to meet applicable standards and/or regulations.

An example mobile testing and evaluation vehicle includes multiple components that are described herein, where a component (or portion thereof) can be made from a single piece (as from a mold or an extrusion). When a component (or portion thereof) of an example mobile testing and evaluation vehicle is made from a single piece, the single piece can be cut out, bent, stamped, and/or otherwise shaped to create certain features, elements, or other portions of the component. Alternatively, a component (or portion thereof) of an example mobile testing and evaluation vehicle can be made from multiple pieces that are mechanically coupled to each other. In such a case, the multiple pieces can be mechanically coupled to each other using one or more of a number of coupling methods, including but not limited to adhesives, welding, fastening devices, compression fittings, mating threads, and slotted fittings. One or more pieces that are mechanically coupled to each other can be coupled in one or more of a number of ways, including but not limited to fixedly, hingedly, rotatably, removably, slidably, and threadably.

Each component and/or feature described herein (including each component of an example mobile testing and evaluation vehicle) can be made of one or more of a number of suitable materials, including but not limited to metal (e.g., stainless steel), ceramic, rubber, glass, and plastic. An example mobile testing and evaluation vehicle can be designed to comply with certain standards and/or requirements. Examples of entities that set such standards and/or requirements can include, but are not limited to, the Society of Petroleum Engineers (SPE), the American Petroleum Institute (API), the International Standards Organization (ISO), and the Occupational Safety and Health Administration (OSHA).

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if an item is described herein as including a component of type A, a component of type B, a component of type C, or any combination thereof, it is understood that this phrase describes all of the various individual and collective combinations and permutations of these components. For example, in some embodiments, the item described by this phrase could include only a component of type A. In some embodiments, the item described by this phrase could include only a component of type B. In some embodiments, the item described by this phrase could include only a component of type C. In some embodiments, the item described by this phrase could include a component of type A and a component of type B. In some embodiments, the item described by this phrase could include a component of type A and a component of type C. In some embodiments, the item described by this phrase could include a component of type B and a component of type C. In some embodiments, the item described by this phrase could include a component of type A, a component of type B, and a component of type C. In some embodiments, the item described by this phrase could include two or more components of type A (e.g., A1 and A2). In some embodiments, the item described by this phrase could include two or more components of type B (e.g., B1 and B2). In some embodiments, the item described by this phrase could include two or more components of type C (e.g., C1 and C2). In some embodiments, the item described by this phrase could include two or more of a first component (e.g., two or more components of type A (A1 and A2)), optionally one or more of a second component (e.g., optionally one or more components of type B), and optionally one or more of a third component (e.g., optionally one or more components of type C). In some embodiments, the item described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the item described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

If a component of a figure is described but not expressly shown or labeled in that figure, the label used for a corresponding component in another figure can be inferred to that component. Conversely, if a component in a figure is labeled but is not described, the description for such component can be substantially the same as the description for the corresponding component in another figure. The numbering scheme for the various components in the figures herein is such that each component is a three-digit number or a four-digit number, and corresponding components in other figures have the identical last two digits. For any figure shown and described herein, one or more of the components may be omitted, added, repeated, and/or substituted. Accordingly, embodiments shown in a particular figure should not be considered limited to the specific arrangements of components shown in such figure.

Further, a statement that a particular embodiment (e.g., as shown in a figure herein) does not have a particular feature or component does not mean, unless expressly stated, that such embodiment is not capable of having such feature or component. For example, for purposes of present or future claims herein, a feature or component that is described as not being included in an example embodiment shown in one or more particular drawings is capable of being included in one or more claims that correspond to such one or more particular drawings herein.

Example embodiments of systems for robotic characterization of impacted sites will be described more fully hereinafter with reference to the accompanying drawings, in which example embodiments of systems for robotic characterization of impacted sites are shown. Systems for robotic characterization of impacted sites may, however, be embodied in many different forms (including variations of a marine vessel with a 2-stage tank filling mechanism) and should not be construed as limited to the example embodiments set forth herein. Rather, these example embodiments are provided so that this disclosure will be thorough and complete, and will fully convey the scope of systems for robotic characterization of impacted sites to those of ordinary skill in the art. Like, but not necessarily the same, elements (also sometimes called components) in the various figures are denoted by like reference numerals for consistency.

Terms such as “first”, “second”, “above”, “below”, “inner”, “outer”, “distal”, “proximal”, “end”, “top”, “bottom”, “upper”, “lower”, “side”, “left”, “right”, “front”, “rear”, and “within”, when present, are used merely to distinguish one component (or part of a component or state of a component) from another. This list of terms is not exclusive. Such terms are not meant to denote a preference or a particular orientation unless explicitly stated, and they are not meant to limit embodiments of systems for robotic characterization of impacted sites. In the following detailed description of the example embodiments, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

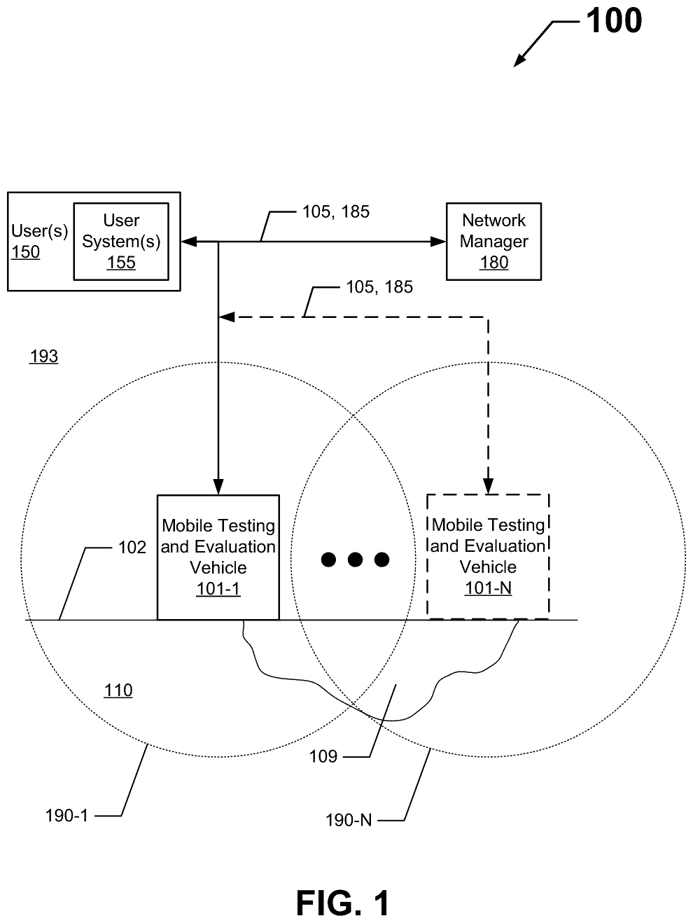

FIG. 1 shows a system 100 for robotically characterizing impacted sites according to certain example embodiments. The system 100 includes multiple components. In this case, the system 100 includes a network manager 180, one or more users 150 (including one or more associated user systems 155), and one or more mobile testing and evaluation vehicles 101 (e.g., mobile testing and evaluation vehicle 101-1, mobile testing and evaluation vehicle 101-N). The one or more mobile testing and evaluation vehicles 101 move along a surface 102. In this case, the surface 102 is a horizontal plane atop the ground 110, and so the mobile testing and evaluation vehicles 101 are exposed to the air 193 (also sometimes called the ambient environment 193 herein). Part of the ground 110 to the surface 102 is impacted with COPC 109 in this case. In alternative embodiments, the surface 102 can be non-planar (e.g., undulating) and/or non-horizontal (e.g., vertical, sloped). The surface 102 can be made of one or more of any of a number of materials, including but not limited to gravel, asphalt, grass, concrete, tile, sand, metal, plastic, and larger rocks.

The system 100 can have any number of mobile testing and evaluation vehicles 101. In this case, the system 100 has N mobile testing and evaluation vehicles 101 (e.g., mobile testing and evaluation vehicle 101-1 through mobile testing and evaluation vehicle 101-N). Each mobile testing and evaluation vehicle 101 has its own zone of interest 190 (ZOI 190), which defines the space in the air 193 and/or the ground 110 that the mobile testing and evaluation vehicle 101 inspects and evaluates. In this case, the mobile testing and evaluation vehicle 101-1 has ZOI 190-1, and the mobile testing and evaluation vehicle 101-N has ZOI 190-N. When the system 100 has multiple mobile testing and evaluation vehicles 101, the ZOI 190 for one mobile testing and evaluation vehicle 101 can be the same as (e.g., in terms of shape, in terms of size, in terms of the ratio of air 193 to ground 110), or different than, the ZOI 190 for one or more of the other mobile testing and evaluation vehicles 101.

Each ZOI 190 can be located indoors and/or outdoors. Each ZOI 190 can be located in any of a number of environments (e.g., climate controlled, humid, hot, cold, corrosive, hazardous, dust-prone). The characteristics (e.g., shape, size, central location) of a ZOI 190 can be based on any of a number of factors, including but not limited to the capabilities (e.g., in terms of the mobility features, in terms of the sensor devices) of the mobile testing and evaluation vehicle 101, the terrain of the surface 102, and the ambient conditions (e.g., in the air 193).

The network manager 180 of the system 100 can be configured to control and/or communicate with one or more other components of the system 100. For example, the network manager 180 may be configured to provide instructions (e.g., an initial starting point, the ZOI 190, tests to perform) to one or more of the mobile testing and evaluation vehicles 101. As another example, the network manager 180 can be configured to receive information (e.g., evaluations, measurements of parameters made by sensor devices (discussed below)) from one or more of the mobile testing and evaluation vehicles 101.

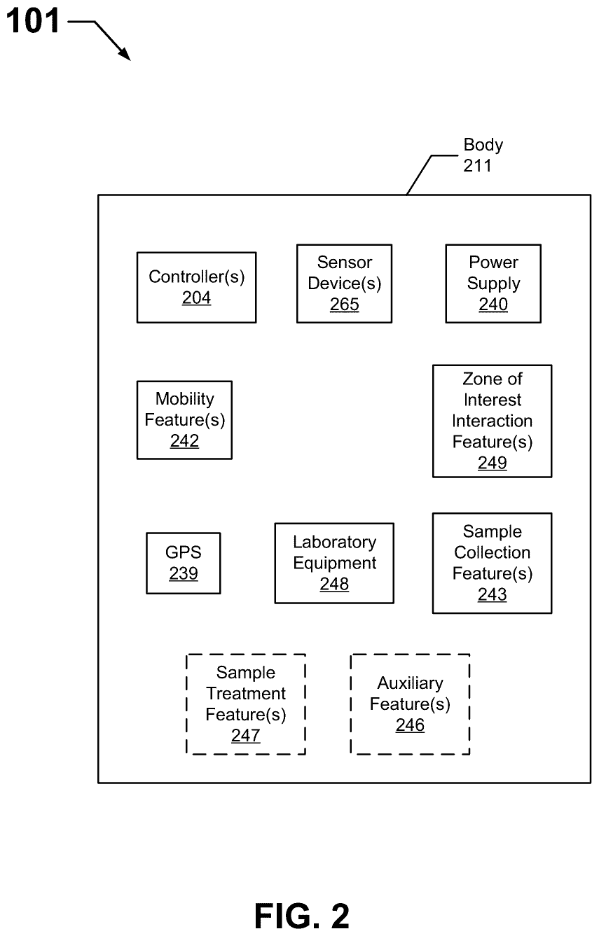

The network manager 180 may be substantially similar to a controller 204, as described below with respect to FIGS. 2 through 4. For example, the network manager 180 may include a controller that has one or more components and/or similar functionality to some or all of the controller 204. Alternatively, the network manager 180 may include one or more of a number of features in addition to, or altered from, the features of the controller 204. As described herein, control and/or communication with the network manager 180 may include communicating with one or more other components of the same system 100 or another system. In such a case, the network manager 180 may facilitate such control and/or communication. The network manager 180 may be called by other names, including but not limited to a master controller, a network controller, and an enterprise manager. The network manager 180 may be considered a type of computer device, as discussed below with respect to FIG. 5.

Each user 150 of the system 100 can be any person or entity that interacts, directly or indirectly, with the network manager 180 and/or one or more of the mobile testing and evaluation vehicles 101, including any portions thereof. Examples of a user 150 may include, but are not limited to, a business owner, a research scientist, an engineer, a company representative, an inspector, a consultant, a government representative, a regulator, a network manager, a contractor, and a manufacturer's representative. A user 150 can use one or more user systems 155, which may include a display (e.g., a GUI). A user system 155 of a user 150 can interact with (e.g., send data to, obtain data from) the network manager 180 (or portions thereof) via an application interface (e.g., application interface 426) and using the communication links 105. The user 150 can also interact directly with the network manager 180 and/or one or more of the mobile testing and evaluation vehicles 101 (including portions thereof) through a user interface (e.g., keyboard, mouse, touchscreen). Examples of a user system 155 can include, but are not limited to, a cell phone, a laptop computer, an electronic tablet, and a specialized handheld device.

As discussed above, the network manager 180 can communicate directly with each of the mobile testing and evaluation vehicles 101. The network manager 180 can also communicate directly with each of the users 150, including any associated user systems 155. Further, each of the mobile testing and evaluation vehicles 101 can communicate directly with each other and/or with each of the users 150, including any associated user systems 155. Such communication can occur using the communication links 105. Each communication link 105 can include wired (e.g., Class 1 electrical cables, Class 2 electrical cables, electrical connectors, Power Line Carrier, RS485) and/or wireless (e.g., Wi-Fi, Zigbee, visible light communication, cellular networking, satellite, Bluetooth, WirelessHART, ISA100) technology. A communication link 105 can be used for the transmission of signals (e.g., communication signals, control signals, data) between the user systems 155, the network manager 180, and the mobile testing and evaluation vehicles 101 (including portions thereof) in the system 100.

In addition, or in the alternative, the network manager 180, the user systems 155, and the mobile testing and evaluation vehicles 101 can have one or more power transfer signals flow therebetween using one or more power transfer links 185. Each power transfer link 185 may include one or more electrical conductors, which may be individual or part of one or more electrical cables. In some cases, as with inductive power, power may be transferred wirelessly using power transfer links 185. Each power transfer link 185 may be sized (e.g., 12 gauge, 18 gauge, 4 gauge) in a manner suitable for the amount (e.g., 480V, 24V, 120V) and type (e.g., alternating current, direct current) of power transferred therethrough.

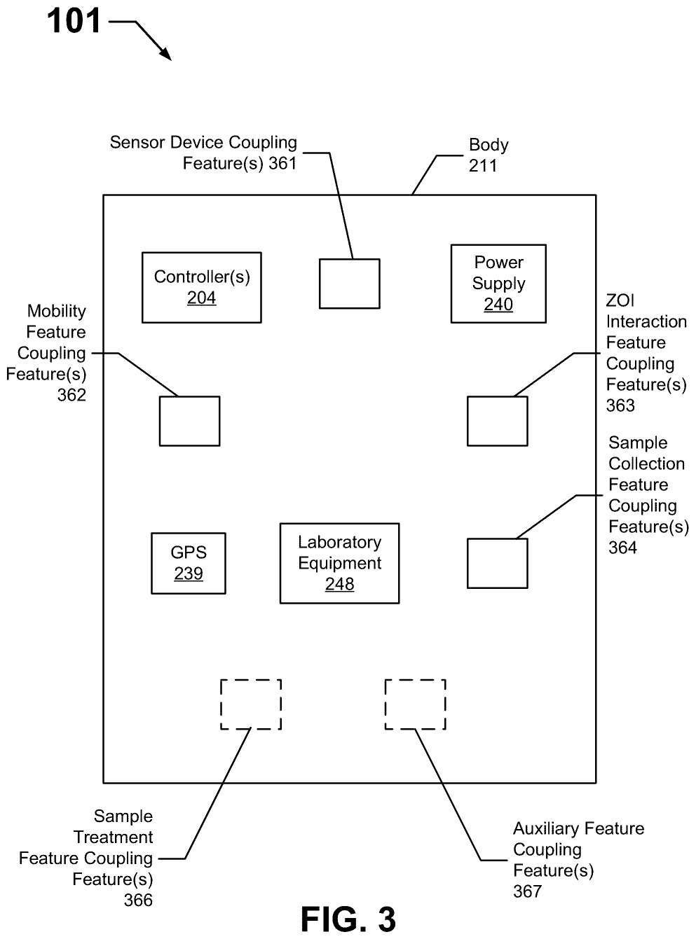

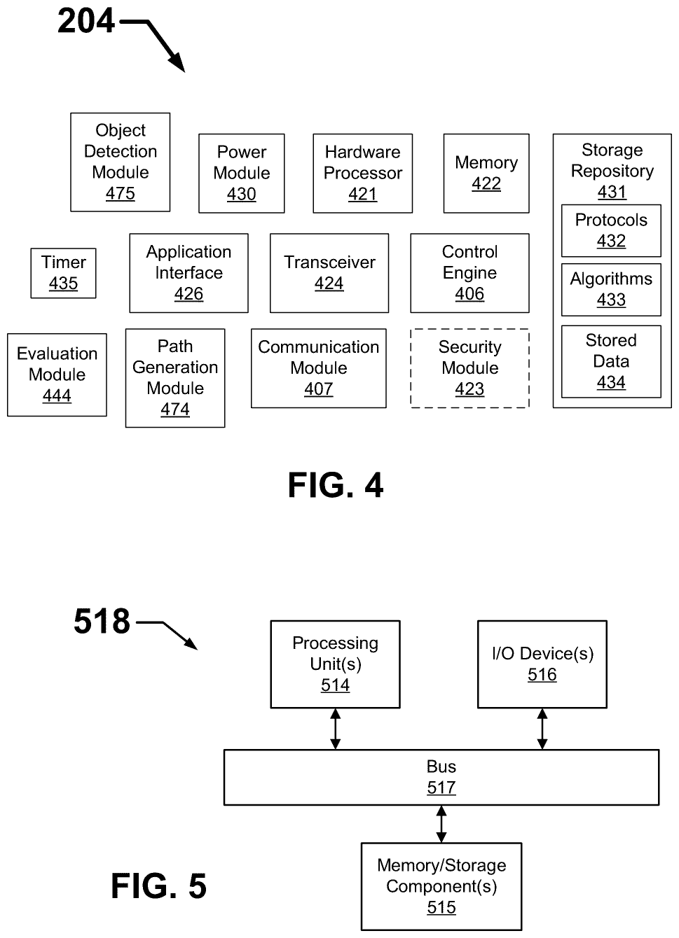

FIG. 2 shows a system diagram of a mobile testing and evaluation vehicle 101 of FIG. 1 according to certain example embodiments. FIG. 3 shows a system diagram of a modular version of the mobile testing and evaluation vehicle 101 of FIG. 2 according to certain example embodiments. FIG. 4 shows a system diagram of a controller 204 of the mobile testing and evaluation vehicle 101 of FIGS. 2 and 3 according to certain example embodiments. Referring to FIGS. 1 through 4, the mobile testing and evaluation vehicle 101 of FIG. 2 can include one or more controllers 204, a power supply 240, one or more sensor devices 265, one or more ZOI interaction features 249, one or more optional mobility features 242, one or more sample collection features 243, a global positioning system 239 (GPS 239), one or more auxiliary features 246, one or more optional sample treatment features 247, a body 211, and laboratory equipment 248.

In some cases, as shown in FIG. 3, the mobile testing and evaluation vehicle 101 can have one or more modular features that allow various portions of the mobile testing and evaluation vehicle 101 to be interchangeable, additive, and/or removable. In this example, the modular version of the mobile testing and evaluation vehicle 101 includes the body 211, the one or more controllers 204, the power supply 240, the GPS 239, and the laboratory equipment 248 as shown in FIG. 2. These components are not modular in this case, but in alternative embodiments, one or more of these components could be modular, having their own coupling features.

In addition, the modular version of a mobile testing and evaluation vehicle 101 of FIG. 3 includes one or more sensor device coupling features 361, one or more mobility feature coupling features 362, one or more ZOI interaction feature coupling features 363, one or more sample collection feature coupling features 364, one or more optional sample treatment feature coupling features 366, and one or more optional auxiliary feature coupling features 367. These components are modular in this case, but in alternative embodiments, one or more of these components may not be modular, lacking their own coupling features.

In certain example embodiments, the modular version of a mobile testing and evaluation vehicle 101 has certain coupling features (e.g., a mobility feature coupling feature 362, a sensor device coupling feature 361, a sample collection feature coupling feature 364) that can universally accept different versions of the feature it is designed to receive. For example, a ZOI interaction feature coupling feature 364 of a mobile testing and evaluation vehicle 101 can be configured to couple to a number of different ZOI interaction features 249 (e.g., one in the form of a back hoe, another in the form of a drill, yet another in the form of an auger, still another in the form of a rake). In this way, if it is determined (e.g., by the controller 204 or portion thereof) that the mobile testing and evaluation vehicle 101 does not have an appropriate ZOI interaction feature 249 for a particular ZOI 190, a user 150 can replace an unneeded version (e.g., a rototiller) of a ZOI interaction feature 249 with a needed version (e.g., a backhoe) of a ZOI interaction feature 249.

Example embodiments can include an integrated robotic platform that can effectively delineate site (e.g., soil, tanks, vessels, concrete pads, roads, platforms, tables, ceilings) impacts (e.g., contamination) by measuring (e.g., using one or more sensor devices 265) contaminant concentrations directly at their locations (e.g., on the surface 102, in the ground 110, in the air 193) within the ZOI 190, without the creation of waste and with limited or no need for human assistance. An example mobile testing and evaluation vehicle 101 can collect samples and/or take measurements autonomously, semi-autonomously, or through remote operation. An example mobile testing and evaluation vehicle 101 can identify the distribution of the COPC 109 without the creation of waste or with minimal waste.

An example mobile testing and evaluation vehicle 101 can include a site-specific portable analyzer (e.g., a controller 204, including portions thereof, with one or more sensor devices 265) that can measure COPC 109 in the ZOI 190 at a level that is adequate to support field decisions. In addition, or in the alternative, an example mobile testing and evaluation vehicle 101 can include one or more of a number of mobility features 242 that allow navigation through natural terrains within the ZOI 190 and perform in-situ field measurements. In addition, or in the alternative, an example mobile testing and evaluation vehicle 101 can include a controller 204 having, for example, a software package to control the mobile testing and evaluation vehicle 101, collect and process analytical data, and store or broadcast relevant data.

The controller 204, as shown in FIG. 4, includes multiple components. For example, in this case, the controller 204 includes a control engine 406, an object detection module 475, an evaluation module 444, a path generation module 474, a communication module 407, a timer 435, a power module 430, a storage repository 431, a hardware processor 421, memory 422, a transceiver 424, an application interface 426, and a security module 423. The various components of the controller 204 may be centrally located. In addition, or in the alternative, some of the components of the controller 204 may be located remotely from (e.g., in the cloud, at an office building) one or more of the other components of the controller 204. A controller 204 can be a type of computing device discussed below with respect to FIG. 5.

The components shown in FIGS. 2 through 4 are not exhaustive, and in some embodiments, one or more of the components shown in FIGS. 2 through 4 may not be included in the mobile testing and evaluation vehicle 101. For example, if there are multiple mobile testing and evaluation vehicles 101 in a system (e.g., system 100), where each mobile testing and evaluation vehicle 101 has its own controller 204, then one controller 204 can be subservient to the other controller 204. In such cases, the subservient controller 204 can lack some of the components (e.g., an object detection module 475, a path generation module 474) and instead rely on the other controller 204 for the capabilities provided by those components.

The body 211 of a mobile testing and evaluation vehicle 101 can include one or more walls that house and/or have disposed thereon one or more of the components of the mobile testing and evaluation vehicle 101. For example, the body 211 can house the controller 204 and have the various coupling features (e.g., the mobility feature coupling features 362, the ZOI interaction feature coupling features 363) disposed thereon. Each component of the mobile testing and evaluation vehicle 101 can be located within the body 211, on the body 211, and/or remotely from the body 211. The body 211 can be configured in such a way that allows the mobile testing and evaluation vehicle 101 to comply with applicable standards for the environment in the ZOI 190.

For example, if the ZOI 190 is in a hazardous environment, the body 211 can include flame paths and/or other appropriate features to allow the body 211 to meet the standards for an explosion-proof enclosure. As other non-exclusive examples, the body 211 can be submersible, water-proof, dust-proof, and/or intrinsically safe. As a specific example, an amphibious platform of the mobile testing and evaluation vehicle 101 can be configured for movement along land and water, particularly for characterizing potentially impacted bodies of water (e.g., in wetlands, in swampy areas). In such embodiments, the mobile testing and evaluation vehicle 101 may be outfitted with a sensor device 265 in the form of a camera that is configured to detect color differences at the surface of the body of water to identify potentially impacted locations. In such cases, the amphibious platform can be or include an amphibious vehicle or a boat.

The one or more ZOI interaction features 249 of the mobile testing and evaluation vehicle 101 can be coupled to (e.g., integrated with) the body 211. Each ZOI interaction feature 249 can be configured to manipulate (e.g., rake the surface 102, dig into the ground 110 through the surface 102, circulate the air 193, add a chemical to the surface 102, the ground 110, and/or the air) the ZOI 190 for the mobile testing and evaluation vehicle 101. Each ZOI interaction feature 249 can be controlled by the controller 204. Examples of a ZOI interaction feature 249 can include, but are not limited to, a shovel, a rake, a back hoe, a fan, a sprayer, an auger, a rototiller, a drill, and an aerator.

In some cases, as with FIG. 3 where parts of a mobile testing and evaluation vehicle 101 is modular, the body 211 can include one or more ZOI interaction feature coupling features 363. Each ZOI interaction feature coupling feature 363 can be configured to allow a ZOI interaction feature 249 to become coupled to and/or decoupled from the body 211. Each ZOI interaction feature coupling feature 363 can be configured to maintain its coupling to the corresponding ZOI interaction feature 249 for as long as the ZOI interaction feature 249 operates. Each ZOI interaction feature coupling feature 363 can provide mechanical, electrical, and communication coupling with respect to the ZOI interaction feature 249.

The one or more sample collection features 243 of a mobile testing and evaluation vehicle 101 can be coupled to (e.g., integrated with) the body 211. Each sample collection feature 243 can be configured to directly collect one or more samples from the air 193, the surface 102, and/or the ground 110 that is within the ZOI 190 for the mobile testing and evaluation vehicle 101. In addition, or in the alternative, the one or more sample collection features 243 of the mobile testing and evaluation vehicle 101 can be configured to indirectly collect one or more samples from one or more of the ZOI interaction features 249. Each sample collection feature 243 can be configured to collect a sample that is in solid form, in liquid form, in gaseous form, or any suitable combination thereof. In some cases, a sample collection feature 243 can collect a sample while preserving the in situ conditions (e.g., pressure, temperature) of the sample. Each sample collection feature 243 can be controlled by the controller 204. Examples of a sample collection feature 243 can include, but are not limited to, a core retriever, a scoop, a shovel, a column, a test tube, a chamber, a bag, and a syringe.

In some cases, as with FIG. 3 where parts of a mobile testing and evaluation vehicle 101 is modular, the body 211 can include one or more sample collection feature coupling features 364. Each sample collection feature coupling feature 364 can be configured to allow a sample collection feature 243 to become coupled to and/or decoupled from the body 211. Each sample collection feature coupling feature 364 can be configured to maintain its coupling to the corresponding sample collection feature 243 for as long as the sample collection feature 243 operates. Each sample collection feature coupling feature 364 can provide mechanical, electrical, and communication coupling with respect to the sample collection feature 243.

The laboratory equipment 248 of a mobile testing and evaluation vehicle 101 can be configured to provide equipment needed for another component (e.g., the sample treatment feature 247, the evaluation module 444 of the controller 204) to perform a function with respect to a sample, whether the sample is collected by a sample collection feature 243 or measured in situ (e.g., in the air 193, in the ground 110, on the surface 102) within the ZOI 190. In other words, the laboratory equipment 248 provides support to one or more other components of the mobile testing and evaluation vehicle 101. Examples of laboratory equipment 248 can include, but is not limited to, a centrifuge, a heater, a cooler, a reactant chamber, a mixer, a pressurization chamber, and an injection mechanism. Some or all of the laboratory equipment 248 can be employed and utilized by the controller 204. In certain example embodiments, the laboratory equipment 248 can also be configured to include features (e.g., conveyance systems, delivery systems) that receive, handle, and subsequently deliver samples with respect to the laboratory equipment 248.

The global positioning system 239 (GPS 239) of a mobile testing and evaluation vehicle 101 can be configured to identify the location of the mobile testing and evaluation vehicle 101 within the ZOI 190. The GPS 239 can be configured to identify the location of the mobile testing and evaluation vehicle 101 in two dimensions (X-Y plane (e.g., along the surface 102)) or in three dimensions (X-Y-Z space) at any point in time. The timer 435 can be used to associate the location of the mobile testing and evaluation vehicle 101 with a time. The GPS 239 can identify the location of the mobile testing and evaluation vehicle 101 continuously, in fixed increments, upon the occurrence of an event (e.g., a measurement made by a sensor device 265, a sample collected by a sample collection feature 243, operation of a mobility feature 242), and/or based on some other factor.

The one or more optional sample treatment features 247 of a mobile testing and evaluation vehicle 101 can be configured to treat a sample before the measurement of a parameter associated with the sample is obtained. The sample treatment features 247 can use the laboratory equipment to perform its function. One or more of the sample treatment features 247 can be controlled by the controller 204. A treatment performed or overseen by a sample treatment feature 247 can include, but is not limited to, heating the sample, cooling the sample, drying the sample, isolating one or more components of the sample, adding a chemical component to the sample, and pressurizing the sample. For example, a sample treatment feature 247 in the form of a heater can heat a sample so that one or more sensor devices 265 that use a sniffing technology can analyze the vapors that result from the heated sample.

In some cases, as with FIG. 3 where parts of a mobile testing and evaluation vehicle 101 is modular, the body 211 can include one or more sample treatment feature coupling features 366. Each sample treatment feature coupling feature 366 can be configured to allow a sample treatment feature 247 to become coupled to and/or decoupled from the body 211. Each sample treatment feature coupling feature 366 can be configured to maintain its coupling to the corresponding sample treatment feature 247 for as long as the sample treatment feature 247 operates. Each sample treatment feature coupling feature 366 can provide mechanical, electrical, and communication coupling with respect to the sample treatment feature 247.

The one or more auxiliary features 246 of a mobile testing and evaluation vehicle 101 can be configured to assist one or more other components of the mobile testing and evaluation vehicle 101 to perform its function. The assistance provided by an auxiliary feature 246 can be mechanical in nature, hydraulic in nature, electrical in nature, and/or of any other discipline. An example of an auxiliary feature 246 is an anchor that secures itself into the ground 110 to stabilize the body 211, as when a ZOI interaction feature 249 in the form of an auger is positioned at the opposite end of the body 211 and used to penetrate the ground 110. Another example of an auxiliary feature 246 is a hydraulic lift that raises and/or lowers the body relative to the surface 102.

Yet another example of an auxiliary feature 246 is equipment that can be used to clean a sensor device 265, including portions thereof. As a specific example, the lens of an IR detector or a XRF detector (both forms of sensor devices 265), when covered in whole or in part with mud, dirt, and/or other debris, can generate erroneous readings. To avoid this situation, one or more auxiliary features 246 (e.g., a movable arm with a cloth, a blower, a directional spray nozzle, a wiper, a movable cover that covers the sensitive portion of the sensor device 265 when the sensor device 265 is not in use) can be used to clean (e.g., periodically, after each use, when readings fall outside a range of expected values) the lens and/or other features of a sensor device 265 that are used to take measurements of one or more parameters. In addition, or in the alternative, the controller 204 of a mobile testing and evaluation vehicle 101 can be configured to test the functionality of a sensor device 265 by instructing the sensor device 265 to measure a parameter of a control (e.g., measure a portion of the ZOI 190 or take a measurement from a portion of the ZOI 190 having a known value of a parameter, measure a known reference on the mobile testing and evaluation vehicle 101).

In some cases, as with FIG. 3 where parts of a mobile testing and evaluation vehicle 101 is modular, the body 211 can include one or more auxiliary feature coupling features 367. Each auxiliary feature coupling feature 367 can be configured to allow an auxiliary feature 246 to become coupled to and/or decoupled from the body 211. Each auxiliary feature coupling feature 367 can be configured to maintain its coupling to the corresponding auxiliary feature 246 for as long as the auxiliary feature 246 operates. Each auxiliary feature coupling feature 367 can provide mechanical, electrical, and communication coupling with respect to the auxiliary feature 246.

The mobility features 242 of a mobile testing and evaluation vehicle 101 can be configured to allow the mobile testing and evaluation vehicle 101, or portions thereof, to move. The mobile testing and evaluation vehicle 101 can have one or more of any number and/or type of mobility features 242. Examples of such mobility features 242 can include, but are not limited to, wheels (e.g., rubber wheels, magnetic wheels), propellers, caterpillar tracks, legs, grippers, suction cups, a crane, a crawler, an extending portion, a retractable portion, anchors, and an elevator. Mobility features 242 can also include other equipment that move any of the above features. Such equipment can include, but is not limited to, motors, axels, gears, a heat sink, an electrical conductor or electrical cable, a terminal block, a drive train, and a circuit board. In this way, the mobile testing and evaluation vehicle 101 can move within the ZOI 190 (e.g., along the surface 102, through the air 193, in the ground 110, in water, up and down stairs, up and down ladders, through doorways and hatchways, up and down a wall, and along a ceiling). In certain example embodiments, a mobility feature 242 can be configured to move the body 211 without substantially disturbing the ZOI 190 or portions thereof.

The power supply 240 of a mobile testing and evaluation vehicle 101 can be configured to provide power to one or more of the other components of the mobile testing and evaluation vehicle 101. The power supply 240 of the mobile testing and evaluation vehicle 101 can include one or more components (e.g., a transformer, a diode bridge, an inverter, a converter) that receives power (for example, through an electrical cable) from a source (e.g., a battery, an electrical generator) and generates power of a type (e.g., alternating current, direct current) and level (e.g., 12V, 24V, 120V) that can be used by one or more of the other components (e.g., the controller 204, the mobility features 242) of a mobile testing and evaluation vehicle 101. In addition, or in the alternative, the power supply 240 can be or include a source of power in itself. For example, the power supply 240 can be or include a battery or some other source of independent power. In some cases, the controller 204 can generate and send a signal to the power supply 240 to control the operation and/or output of the power supply 240.

Each of the sensor devices 265 of a mobile testing and evaluation vehicle 101 is configured to measure one or more parameters that are associated with the ZOI 190 and/or a sample therein. Each sensor device 265 can include one or more sensors that measure one or more parameters (e.g., pressure, flow rate, temperature, thickness, corrosion, gas composition, magnetic field, proximity, radiation, chemical composition, humidity, resistance, moisture, X-ray fluorescence, carbon isotopes, carbon dioxide, nitrogen, phosphorous, potassium, mass, weight, thermal load, fluid level, speed, distance, height, tension) within the ZOI 190.

Examples of a sensor device 265 can include, but are not limited to, a thermometer, a hydrometer, a gas chromatograph, lidar, radar, sonar and/or other forms of ground penetrating radar, a voltmeter, an ammeter, a flow sensor, a pressure sensor, a gas spectrometer, a permeability meter, a porosimeter, an X-ray machine, an X-ray fluorescence (XRF) detector or probe, a Mid Infrared (MIR) sensor, a Near Infrared (NIR) sensor, a general infrared sensor, a radiation/radioactivity counter, an optical microscope, an inverted microscope, a brightfield microscope, a fluorescence microscope, a phase contrast microscope, a Z-stack microscope, a portable gas chromatograph, a portable fluorescence detector, a carbon isotope detector, a CO₂ detector, a nitrogen/phosphorous/potassium (NPK) sensor, a moisture sensor, a mercury vapor sensor, a H₂S sensor, a Volatile Organic Carbons (VOCs) sensor, a Carbon Monoxide (CO) detector, a Carbon Dioxide (CO₂) detector, a Carbon Disulfide (CS₂) detector, a Carbonyl Sulfide (COS) detector, a methane detector, an oxygen sensor, a sensor that detects acidic gases (e.g., hydrogen chloride vapor), a sensor that detects radioactive gases (e.g., radon, thoron, actinon), a metal detector, a Geiger counter, a Polymerase Chain Reactor (PCR) machine (e.g., to detect the presence, type, and/or abundance of soil microorganisms to evaluate soil RNA and/or DNA), and a camera (e.g., configured for hyperspectral imaging). A sensor device 265 can be configured to measure a parameter continuously, at discrete intervals (e.g., every 60 seconds), randomly, on demand (e.g., from the controller 204), and/or on some other basis.

In some embodiments, a sensor device 265 can be or include one or more integrated optical cameras with machine learning image processing for contaminant identification and continuous improvement of data quality. One or more shutters (part of the auxiliary features 246) on the mobile testing and evaluation vehicle 101 may be closed to protect the site-analyzing sensor device 265 when it is not in use. The site-analyzing sensor device 265 and shutter may be connected to the mobile testing and evaluation vehicle 101 by a spring or other device that can mechanically retract the sensor device 265 away from the site surface 102 and close the shutter in the event of a power outage.

A controller 204 of a mobile testing and evaluation vehicle 101 can be configured to control and/or communicate with the other components (e.g., the mobility features 242, the ZOI interaction features 249), or portions thereof, of the mobile testing and evaluation vehicle 101. A controller 204 performs a number of functions that may include receiving data, evaluating data, following protocols, running algorithms, receiving instructions, and sending instructions. A mobile testing and evaluation vehicle 101 can have a single controller 204 or multiple controllers 204. When there are multiple controllers 204 of a mobile testing and evaluation vehicle 101, each controller 204 can operate independently of each other. Alternatively, one or more of the controllers 204 in a mobile testing and evaluation vehicle 101 can work cooperatively with each other. As yet another alternative, one of the controllers 204 of a mobile testing and evaluation vehicle 101 can control some or all of one or more other controllers 204 of a mobile testing and evaluation vehicle 101.

The collection of samples, the evaluation of samples, the generation of paths, the revision of paths, the treatment of a sample, the interaction with a ZOI 190, the measurement of parameters, the identification of objects, the evaluation of a ZOI 190, and all other steps discussed herein may be performed by the mobile testing and evaluation vehicle 101 (in some cases with the assistance of the network manager 180) autonomously without human intervention or control using example embodiments.

The storage repository 431 of a controller 204 of a mobile testing and evaluation vehicle 101 may be a persistent storage device (or set of devices) that stores software and data used to assist the controller 204 in communicating with one or more other components of the mobile testing and evaluation vehicle 101 (e.g., a sensor device 265, a mobility feature 242) and/or other components of the system 100 (e.g., a user system 155, the network manager 180). In one or more example embodiments, the storage repository 431 stores one or more protocols 432, one or more algorithms 433, and stored data 434.

The protocols 432 of the storage repository 431 may be any procedures (e.g., a series of method steps) and/or other similar operational processes that the control engine 406 of the controller 204 follows based on certain conditions at a point in time. The protocols 432 may include any of a number of communication protocols that are used to send and/or obtain data between the controller 204 and other components of a system (e.g., system 100) or portion thereof (e.g., a mobile testing and evaluation vehicle 101). Such protocols 432 used for communication may be a time-synchronized protocol. Examples of such time-synchronized protocols may include, but are not limited to, a highway addressable remote transducer (HART) protocol, a wirelessHART protocol, and an International Society of Automation (ISA) 100 protocol. In this way, one or more of the protocols 432 may provide a layer of security to the data transferred within a system or portion thereof (e.g., a mobile testing and evaluation vehicle 101). Other protocols 432 used for communication may be associated with the use of Wi-Fi, Zigbee, visible light communication (VLC), cellular networking, BLE, UWB, and Bluetooth.

The algorithms 433 may be any formulas, mathematical models, forecasts, simulations, and/or other similar tools that the control engine 406 and/or other component of the controller 204 uses to reach a computational conclusion. For example, one or more algorithms 433 may be used, in conjunction with one or more protocols 432, to assist the controller 204 to equip and deploy a mobile testing and evaluation vehicle 101 to perform a particular function. As another example, one or more algorithms 433 may be used, in conjunction with one or more protocols 432, to assist the controller 204 to receive and interpret a request or instruction from the network manager 180. As yet another example, one or more algorithms 433 may be used, in conjunction with one or more protocols 432, to assist the controller 204 to manage a mobile testing and evaluation vehicle 101 during a function. As yet another example, one or more algorithms 433 may be used, in conjunction with one or more protocols 432, to assist the controller 204 to retrieve measurements (e.g., data) collected by a sensor device 265 during performance of a function. An example of one or more algorithms 432 can be or include one or more statistical (e.g., Gaussian) reasoning functions.

Stored data 434 may be any data associated with the other components (e.g., a user system 155, the network manager 180) of a system (e.g., system 100), the environment surrounding the ZOI 190, the various components (e.g., the mobility features 242, the sensor devices 265), including associated equipment (e.g., motors, pumps, compressors), of the mobile testing and evaluation vehicle 101, values associated with measurements made by the sensor devices 265, threshold values, tables, results of previously run or calculated algorithms 433, updates to protocols 432, user preferences, and/or any other suitable data. Such data may be any type of data, including but not limited to historical data, present data, and future data (e.g., forecasts). The stored data 434 may be associated with some measurement of time derived, for example, from the timer 435.

Examples of a storage repository 431 may include, but are not limited to, a database (or a number of databases), a file system, cloud-based storage, a hard drive, flash memory, some other form of solid-state data storage, or any suitable combination thereof. The storage repository 431 may be located on multiple physical machines, each storing all or a portion of the protocols 432, the algorithms 433, and/or the stored data 434 according to some example embodiments. Each storage unit or device may be physically located in the same or in a different geographic location.

The storage repository 431 may be operatively connected to the control engine 406. In one or more example embodiments, the control engine 406 includes functionality to communicate with the various components (e.g., the sensor devices 265, the sample collection features 243, the mobility features 242, the ZOI interaction features 249) of the mobile testing and evaluation vehicle 101 and/or the other components (e.g., the network manager 180, the user systems 155, another mobile testing and evaluation vehicle 101) of the system 100. More specifically, the control engine 406 sends information to and/or obtains information from the storage repository 431 in order to communicate with the various components of a mobile testing and evaluation vehicle 101 and/or the various components of the system 100. As discussed below, the storage repository 431 may also be operatively connected to the communication module 407 in certain example embodiments.

In certain example embodiments, the control engine 406 of the controller 204 controls the operation of one or more components (e.g., the communication module 407, the timer 435, the transceiver 424) of the controller 204. For example, the control engine 406 may activate the communication module 407 when the communication module 407 is in “sleep” mode and when the communication module 407 is needed to send data obtained from another component (e.g., a sensor device 265) or another controller (e.g., a controller 204 of another mobile testing and evaluation vehicle 101) in the system 100. In addition, the control engine 406 of the controller 204 may control the operation of one or more other components (e.g., a controller 204 of another mobile testing and evaluation vehicle 101), or portions thereof, of the system 100.

The control engine 406 of the controller 204 may communicate with one or more other components of the mobile testing and evaluation vehicle 101. For example, the control engine 406 may use one or more protocols 432 to facilitate communication with the sensor devices 265 to obtain data (e.g., measurements of various parameters), whether in real time or on a periodic basis and/or to instruct a sensor device 265 to take a measurement. The control engine 406 may use measurements of parameters taken by sensor devices 265 to evaluate one or more samples taken from a ZOI 190.

As yet another example, one or more algorithms 433 may be used, in conjunction with one or more protocols 432, to assist the controller 204 to evaluate the ZOI 190 based on evaluating the samples. As still another example, one or more algorithms 433 may be used, in conjunction with one or more protocols 432, to assist the controller 204 to control the operation of one or more components (e.g., the ZOI interaction features 249, the mobility features 242, the auxiliary features 246, the sample treatment features 247, the sample collection features 243, the laboratory equipment 248) of the mobile testing and evaluation vehicle 101.

In certain example embodiments, the control engine 406 of the controller 204 of a mobile testing and evaluation vehicle 101, using one or more protocols 432, one or more algorithms 433, and/or stored date 434, can be configured to ensure that each sensor device 265 is operating properly. For example, the control engine 406, following one or more of the protocols 432, can be configured to move (e.g., after the measurement of a certain number (e.g., one, three, six) of samples, after a certain amount of time) the mobile testing and evaluation vehicle 101 toward a known location in the ZOI 190 with a clean surface material (a control) to determine whether or not any contamination of the sensor device 265 exists by comparing the actual reading of the control with an expected value or range of expected values.

As another example, the mobile testing and evaluation vehicle 101 can include an auxiliary feature 246 in the form of a control (e.g., positioned vertically near the sensor device 265, normally positioned upside down to stay clean and movable to be measured by the sensor device 265 to prevent material falling onto the control or sticking to the control). The control can be easily accessible for the sensor device 265 to measure so the controller 204 can decide if contamination is present above an acceptable level. If action is needed to clean the control to ensure that an unacceptable reading is not due to contamination on the sensor device 265, another auxiliary feature 246 (e.g., in the form of a wet sponge at the end of a movable arm, in the form of a water spray nozzle) can be controlled by the controller 204 to clean the portion(s) of the sensor device 265 used to measure a parameter.

As yet another example, the controller 204 can control the mobile testing and evaluation vehicle 101 to move (e.g., after some number (e.g., one, three, seven) of measurements, after failing to clean the sensor device 265 in situ using one or more auxiliary features 246) to a cleaning station in or near the ZOI 190 to clean the sensor device. Such a cleaning station can include, for example, a water jet system that filters and recycles its rinse water. The cleaning station can run continuously, when sensing (e.g., using its own sensor device 265) an object approach, when instructed by the controller 204 of the mobile testing and evaluation vehicle 101, and/or on some other basis.

As still another example, the sensor of the sensor device 265 can be protected by a film that is easily removable and does not (significantly) interfere with the readings of the sensor service 265. For instance, the mobile testing and evaluation vehicle 101 can include an auxiliary feature 246 in the form of a roll of plastic foil or film, which can be installed next to a sensor of the sensor device 265. In such a case, the roll can be extended on some basis (e.g., after every measurement, when the sensor fails a clean test) so that a new piece of plastic is moved in front of the sensor. Alternatively, multiple frames with appropriate clean foil could be installed to be switched in front of the sensor on some basis (e.g., after each sample, after a pre-determined number of samples, after the sensor fails a clean test).

The control engine 406 may generate and process data associated with control, communication, and/or other signals sent to and obtained from another component (e.g., a sensor device 265, another controller (e.g., a controller 204 of another mobile testing and evaluation vehicle 101, the network manager 180, a user system 155) of the system 100. In certain embodiments, the control engine 406 of the controller 204 may communicate with one or more components of a system external to the system 100. For example, the control engine 406 may interact with an inventory management system by ordering replacements for components or pieces of equipment (e.g., a sensor device 265, a valve, a motor) within the mobile testing and evaluation vehicle 101 that has failed or is failing. As another example, the control engine 406 may interact with a contractor or workforce scheduling system by arranging for the labor needed to replace a component or piece of equipment in the mobile testing and evaluation vehicle 101. In this way and in other ways, the controller 204 is capable of performing a number of functions beyond what could reasonably be considered a routine task.

In certain example embodiments, the control engine 406 may include an interface that enables the control engine 406 to communicate with another component (e.g., a sensor device 265, another controller (e.g., a controller 204 of another mobile testing and evaluation vehicle 101, the network manager 180, a user system 155) of the system 100. For example, if a controller 204 of another mobile testing and evaluation vehicle 101 operates under IEC Standard 62386, then the controller 204 of the other mobile testing and evaluation vehicle 101 may have a serial communication interface that will transfer data to the controller 204. Such an interface may operate in conjunction with, or independently of, the protocols 432 used to communicate between the controller 204 and the other components of the system 100.

The control engine 406 (or other components of the controller 204) may also include one or more hardware components and/or software elements to perform its functions. Such components may include, but are not limited to, a universal asynchronous receiver/transmitter (UART), a serial peripheral interface (SPI), a direct-attached capacity (DAC) storage device, an analog-to-digital converter, an inter-integrated circuit (I2C), and a pulse width modulator (PWM).

The evaluation module 444 of the controller 204 of a mobile testing and evaluation vehicle 101 may be configured to evaluate one or more samples and/or the ZOI 190 using data and/or communication signals received from another component of the mobile testing and evaluation vehicle 101. In some cases, the evaluation module 444 may be configured to operate based on an instruction received from the control engine 406. The evaluation module 444 can be configured to evaluate one or more of the samples (whether collected or not) using the measurements made by the sensor devices 265.

In certain example embodiments, the evaluation module 444 can instruct one or more of the sample treatment features 247 to treat one or more samples so that the evaluation module 444 can receive additional information to evaluate the samples. The evaluation module 444 can also be configured to evaluate the ZOI 190 (and, more specifically, the COPC 109 within the ZOI 190) based on the evaluations of the samples. In some cases, the evaluation module 444 can be used to validate or refute prior testing (e.g., a bioremediation screening) performed by an entity (e.g., a user 150) other than the mobile testing and evaluation vehicle 101.

The object detection module 475 of a mobile testing and evaluation vehicle 101 may be configured to recognize one or more objects within the ZOI 190. In some cases, an object can prevent the mobile testing and evaluation vehicle 101 from traveling therethrough. Objects of this type can vary based on one or more of a number of factors, including but not limited to the types of sensor devices 265 on board the mobile testing and evaluation vehicle 101 and the capability of the mobility features 242 of the mobile testing and evaluation vehicle 101. Examples of such an object can include, but are not limited to, one or more boulders, a building or other structure, a body of water, mud, a tar pit, and quicksand.

In other cases, an object can present a danger to the mobile testing and evaluation vehicle 101 and/or other beings in or near the ZOI 190. Examples of such an object can include, but are not limited to, a landmine and an improvised explosive device. The object detection module 475 can be configured to use measurements made by one or more sensor devices 265 to identify, both in terms of location and in terms of type, one or more objects in the ZOI 190. The conclusions reached by the object detection module 475 can be provided to and used by the path generation module 474. The object detection module 475 can use one or more algorithms 433, one or more protocols 432, and/or stored data 434 to locate and identify an object.

The path generation module 474 of a mobile testing and evaluation vehicle 101 may be configured to generate and/or modify a path by which the mobile testing and evaluation vehicle 101 travels within the ZOI 190 while evaluating the samples and/or the ZOI 190. The path generation module 474 can generate and/or modify a path based on one or more measurements made by one or more of the sensor devices 265, information provided by the object detection module 475, and/or information provided by the evaluation module 444. The path generation module 474 can use one or more algorithms 433, one or more protocols 432, and/or stored data 434 to generate and/or modify a path of travel for the mobile testing and evaluation vehicle 101 within the ZOI 190.

The communication module 407 of the controller 204 determines and implements the communication protocol (e.g., from the protocols 432 of the storage repository 431) that is used when the control engine 406 communicates with (e.g., sends signals to, obtains signals from) the other components (e.g., the sensor devices 265, the evaluation module 444, the mobility features 242) of the mobile testing and evaluation vehicle 101 and the other components (e.g., the network manager 180, a user system 155, another mobile testing and evaluation vehicle 101) of the system 100. In some cases, the communication module 407 accesses the stored data 434 to determine which communication protocol is used to communicate with another component of the system 100. In addition, the communication module 407 may identify and/or interpret the communication protocol of a communication obtained by the controller 204 so that the control engine 406 may interpret the communication. The communication module 407 may also provide one or more of a number of other services with respect to data sent from and obtained by the controller 204. Such services may include, but are not limited to, data packet routing information and procedures to follow in the event of data interruption.

The timer 435 of the controller 204 may track clock time, intervals of time, an amount of time, and/or any other measure of time. The timer 435 may also count the number of occurrences of an event, whether with or without respect to time. Alternatively, the control engine 406 may perform a counting function. The timer 435 is able to track multiple time measurements and/or count multiple occurrences concurrently. The timer 435 may track time periods based on an instruction obtained from the control engine 406, based on an instruction obtained from the network manager 180, based on an instruction programmed in the software for the controller 204, based on some other condition (e.g., the occurrence of an event) or from some other component, or from any combination thereof. In certain example embodiments, the timer 435 may provide a time stamp for each packet of data obtained from another component (e.g., a sensor device 265) of the system 100.

The power module 430 of the controller 204 obtains power from a power supply (e.g., the power supply 240) and manipulates (e.g., transforms, rectifies, inverts) that power to provide the manipulated power to one or more other components (e.g., the timer 435, the control engine 406) of the controller 204, where the manipulated power is of a type (e.g., alternating current, direct current) and level (e.g., 12V, 24V, 120V) that may be used by the other components of the controller 204. In some cases, the power module 430 may also provide power to one or more of the sensor devices 265.

The power module 430 may include one or more of a number of single or multiple discrete components (e.g., transistor, diode, resistor, transformer) and/or a microprocessor. The power module 430 may include a printed circuit board, upon which the microprocessor and/or one or more discrete components are positioned. In addition, or in the alternative, the power module 430 may be a source of power in itself to provide signals to the other components of the controller 204. For example, the power module 430 may be or include an energy storage device (e.g., a battery). As another example, the power module 430 may be or include a localized photovoltaic power system.

The hardware processor 421 of the controller 204 executes software, algorithms (e.g., algorithms 433), and firmware in accordance with one or more example embodiments. Specifically, the hardware processor 421 may execute software on the control engine 406 or any other portion of the controller 204, as well as software used by the controller of another mobile testing and evaluation vehicle 101, the network manager 180, and/or other components of the system 100. The hardware processor 421 may be an integrated circuit, a central processing unit, a multi-core processing chip, SoC, a multi-chip module including multiple multi-core processing chips, or other hardware processor in one or more example embodiments. The hardware processor 421 may be known by other names, including but not limited to a computer processor, a microprocessor, and a multi-core processor.

In one or more example embodiments, the hardware processor 421 executes software instructions stored in memory 422. The memory 422 includes one or more cache memories, main memory, and/or any other suitable type of memory. The memory 422 may include volatile and/or non-volatile memory. The memory 422 may be discretely located within the controller 204 relative to the hardware processor 421. In certain configurations, the memory 422 may be integrated with the hardware processor 421.

In certain example embodiments, the controller 204 does not include a hardware processor 421. In such a case, the controller 204 may include, as an example, one or more field programmable gate arrays (FPGA), one or more insulated-gate bipolar transistors (IGBTs), and/or one or more integrated circuits (ICs). Using FPGAs, IGBTs, ICs, and/or other similar devices known in the art allows the controller 204 (or portions thereof) to be programmable and function according to certain logic rules and thresholds without the use of a hardware processor. Alternatively, FPGAs, IGBTs, ICs, and/or similar devices may be used in conjunction with one or more hardware processors 421.

The transceiver 424 of the controller 204 may send and/or obtain control and/or communication signals. Specifically, the transceiver 424 may be used to transfer data between the controller 204 and the other components (e.g., the sensor devices 265, the evaluation module 444, the mobility features 242) of the mobile testing and evaluation vehicle 101 and the other components (e.g., the network manager 180, a user system 155, another mobile testing and evaluation vehicle 101) of the system 100. The transceiver 424 may use wired and/or wireless technology. The transceiver 424 may be configured in such a way that the control and/or communication signals sent and/or obtained by the transceiver 424 may be obtained and/or sent by another transceiver that is part of a component (e.g., the sensor devices 265, the evaluation module 444, the mobility features 242) of the mobile testing and evaluation vehicle 101 and/or another component (e.g., the network manager 180, a user system 155, another mobile testing and evaluation vehicle 101) of the system 100. The transceiver 424 may send and/or obtain any of a number of signal types, including but not limited to radio frequency signals.

When the transceiver 424 uses wireless technology, any type of wireless technology may be used by the transceiver 424 in sending and obtaining signals. Such wireless technology may include, but is not limited to, Wi-Fi, Zigbee, VLC, cellular networking, BLE, UWB, and Bluetooth. The transceiver 424 may use one or more of any number of suitable communication protocols (e.g., ISA100, HART) when sending and/or obtaining signals.

Optionally, in one or more example embodiments, the security module 423 secures interactions between the controller 204, the other components (e.g., the sensor devices 265, the evaluation module 444, the mobility features 242) of the mobile testing and evaluation vehicle 101 and the other components (e.g., the network manager 180, a user system 155, another mobile testing and evaluation vehicle 101) of the system 100. More specifically, the security module 423 authenticates communication from software based on security keys verifying the identity of the source of the communication. For example, user software may be associated with a security key enabling the software of the network manager 180 to interact with the controller 204. Further, the security module 423 may restrict receipt of information, requests for information, and/or access to information.

The other components (e.g., the sensor devices 265, the evaluation module 444, the mobility features 242) of the mobile testing and evaluation vehicle 101 and the other components (e.g., the network manager 180, a user system 155, another mobile testing and evaluation vehicle 101) of the system 100 may interact with the controller 204 of the mobile testing and evaluation vehicle 101 using the application interface 426. Specifically, the application interface 426 of the controller 204 obtains data (e.g., information, communications, instructions, updates to firmware, updates to software) from and sends data (e.g., information, communications, instructions, updates to firmware, updates to software) to the other components (e.g., the sensor devices 265, the evaluation module 444, the mobility features 242) of the mobile testing and evaluation vehicle 101 and the other components (e.g., the network manager 180, a user system 155, another mobile testing and evaluation vehicle 101) of the system 100.

Examples of an application interface 426 may be or include, but are not limited to, an application programming interface, a web service, a data protocol adapter, some other hardware and/or software, or any suitable combination thereof. Similarly, the other components (e.g., the sensor devices 265, the evaluation module 444, the mobility features 242) of the mobile testing and evaluation vehicle 101 and the other components (e.g., the network manager 180, a user system 155, another mobile testing and evaluation vehicle 101) of the system 100 may include an interface (similar to the application interface 426 of the controller 204) to obtain data from and send data to the controller 204 in certain example embodiments.

FIG. 5 illustrates one embodiment of a computing device 518 that implements one or more of the various techniques described herein, and which is representative, in whole or in part, of the elements described herein pursuant to certain example embodiments. For example, a controller 204 (including components thereof, such as a control engine 406, a hardware processor 421, a storage repository 431, a power module 430, and a transceiver 424) may be considered a computing device 518. Computing device 518 is one example of a computing device and is not intended to suggest any limitation as to scope of use or functionality of the computing device and/or its possible architectures. Neither should the computing device 518 be interpreted as having any dependency or requirement relating to any one or combination of components illustrated in the example computing device 518.

The computing device 518 includes one or more processors or processing units 514, one or more memory/storage components 515, one or more input/output (I/O) devices 516, and a bus 517 that allows the various components and devices to communicate with one another. The bus 517 represents one or more of any of several types of bus structures, including a memory bus or memory controller, a peripheral bus, an accelerated graphics port, and a processor or local bus using any of a variety of bus architectures. The bus 517 may include wired and/or wireless buses.

The memory/storage component 515 represents one or more computer storage media. The memory/storage component 515 includes volatile media (such as random access memory (RAM)) and/or nonvolatile media (such as read only memory (ROM), flash memory, optical disks, magnetic disks, and so forth). The memory/storage component 515 includes fixed media (e.g., RAM, ROM, a fixed hard drive, etc.) as well as removable media (e.g., a flash memory drive, a removable hard drive, an optical disk, and so forth).

One or more I/O devices 516 allow a user 150 to enter commands and information to the computing device 518, and also allow information to be presented to the user 150 (including an associated user system 155) and/or other components or devices. Examples of input devices 516 include, but are not limited to, a keyboard, a cursor control device (e.g., a mouse), a microphone, a touchscreen, and a scanner. Examples of output devices include, but are not limited to, a display device (e.g., a monitor or projector), speakers, outputs to a lighting network (e.g., DMX card), a printer, and a network card.

Various techniques are described herein in the general context of software or program modules. Generally, software includes routines, programs, objects, components, data structures, and so forth that perform particular tasks or implement particular abstract data types. An implementation of these modules and techniques are stored on or transmitted across some form of computer readable media. Computer readable media is any available non-transitory medium or non-transitory media that is accessible by a computing device. By way of example, and not limitation, computer readable media includes “computer storage media”.

“Computer storage media” and “computer readable medium” include volatile and non-volatile, removable and non-removable media implemented in any method or technology for storage of information such as computer readable instructions, data structures, program modules, or other data. Computer storage media include, but are not limited to, computer recordable media such as RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which is used to store the desired information and which is accessible by a computer.

The computer device 518 (also sometimes called a computer system 518) is connected to a network (not shown) (e.g., a LAN, a WAN such as the Internet, cloud, or any other similar type of network) via a network interface connection (not shown) according to some example embodiments. Those skilled in the art will appreciate that many different types of computer systems exist (e.g., desktop computer, a laptop computer, a personal media device, a mobile device, such as a cell phone or personal digital assistant, or any other computing system capable of executing computer readable instructions), and the aforementioned input and output means take other forms, now known or later developed, in other example embodiments. Generally speaking, the computer system 518 includes at least the minimal processing, input, and/or output means necessary to practice one or more embodiments.

Further, those skilled in the art will appreciate that one or more elements of the aforementioned computer device 518 is located at a remote location and connected to the other elements over a network in certain example embodiments. Further, one or more embodiments is implemented on a distributed system having one or more nodes, where each portion of the implementation (e.g., a mobile testing and evaluation vehicle 101) is located on a different node within the distributed system. In one or more embodiments, the node corresponds to a computer system. Alternatively, the node corresponds to a processor with associated physical memory in some example embodiments. The node alternatively corresponds to a processor with shared memory and/or resources in some example embodiments.

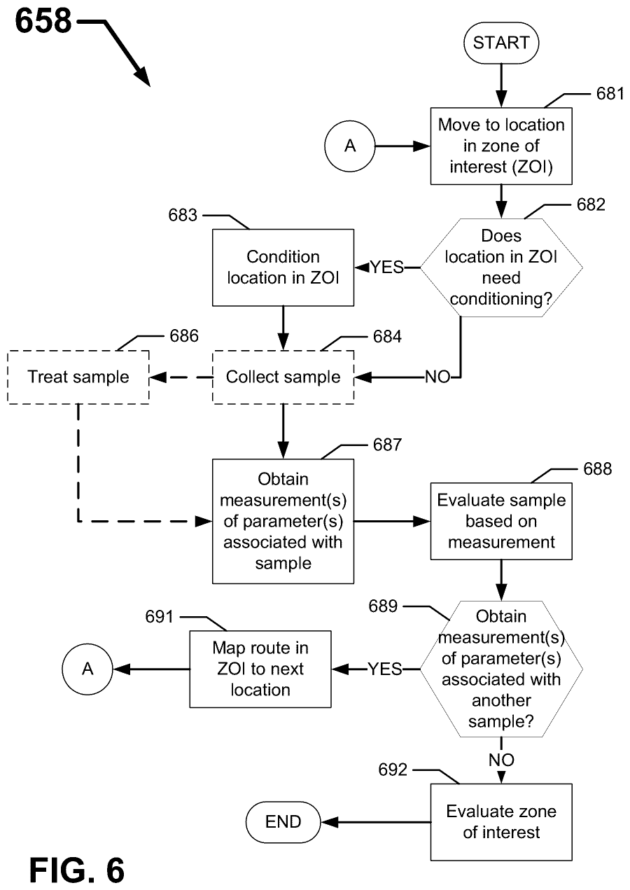

FIG. 6 shows a flowchart 658 of a method for assessing a site with COPC according to certain example embodiments. While the various steps in this flowchart 658 are presented sequentially, one of ordinary skill will appreciate that some or all of the steps may be executed in different orders, may be combined or omitted, and some or all of the steps may be executed in parallel. Further, in one or more of the example embodiments, one or more of the steps shown in this example method may be omitted, repeated, and/or performed in a different order.

In addition, a person of ordinary skill in the art will appreciate that additional steps not shown in FIG. 6 may be included in performing this method. Accordingly, the specific arrangement of steps should not be construed as limiting the scope. Further, a particular computing device, such as the computing device 518 discussed above with respect to FIG. 5, may be used to perform or facilitate performance of one or more of the steps (or portions thereof) for the method shown in FIG. 6 in certain example embodiments. Any of the functions (or portions thereof) performed below by a controller 204 may involve the use of one or more protocols 432, one or more algorithms 433, and/or stored data 434 stored in a storage repository 431. In some cases, one or more of the various steps in the method of FIG. 6 can be performed automatically, as by a controller 204 of a mobile testing and evaluation vehicle 101.

The method shown in FIG. 6 is merely an example that may be performed by using an example system described herein. In other words, systems for assessing a site with COPC may perform other functions using other methods in addition to and/or aside from those shown in FIG. 6. Referring to FIGS. 1 through 6, the method shown in the flowchart 658 of FIG. 6 begins at the START step and proceeds to step 681, where a mobile testing and evaluation vehicle 101 is moved to a location within a ZOI 190. The mobile testing and evaluation vehicle 101 can initially be brought to or near the ZOI 190 by a user 150. The location can be determined using the GPS 239 of the mobile testing and evaluation vehicle 101. The location can be based on a path generated and/or revised by the path generation module 474 of the controller 204.

When the process of evaluating the site with COPC is about to begin or is underway, the controller 204 of the mobile testing and evaluation vehicle 101, with the aid of the path generation module 474, one or more algorithms 433, one or more protocols 432, and/or stored data 434, can control one or more of the mobility features 242 to move the mobile testing and evaluation vehicle 101 to the desired location in the ZOI 190. In certain example embodiments, the mobile testing and evaluation vehicle 101 is moved to the location autonomously and without any human interaction or intervention.

In step 682, a determination is made as to whether the location within the ZOI 190 needs conditioning. In certain example embodiments, the control engine 406 of the controller 204 of the mobile testing and evaluation vehicle 101 can make the determination as to whether conditioning of the location within the ZOI 190 is needed. In such a case, the control engine 406 can use one or more algorithms 433, one or more protocols 432, and/or stored data 434 (e.g., measurements made by one or more of the sensor devices 265) to make the determination. In addition, or in the alternative, the determination can be made by a user 150 communicating with the controller 204 of the mobile testing and evaluation vehicle 101. In certain example embodiments, the determination is made autonomously and without any human interaction or intervention. If the location within the ZOI 190 needs conditioning, then the process proceeds to step 683. If the location within the ZOI 190 does not need conditioning, then the process proceeds to optional step 684 if a sample is collected or otherwise to step 687.

In step 683, the location in the ZOI 190 is conditioned. In certain example embodiments, the location in the ZOI 190 is conditioned by one or more of the ZOI interaction features 249 of the mobile testing and evaluation vehicle 101. In such a case, the one or more of the ZOI interaction features 249 can be controlled by the control engine 406 of the controller 204 of the mobile testing and evaluation vehicle 101 using one or more algorithms 433, one or more protocols 432, and/or stored data 434 (e.g., measurements made by one or more of the sensor devices 265).

The controller 204 of the mobile testing and evaluation vehicle 101, a user 150, and/or the network manager 180 can determine how (e.g., which of the ZOI interaction features 249 is used) the location is conditioned, how long the location is conditions, and/or any other considerations relevant to the conditioning of the location. In certain example embodiments, the location is conditioned autonomously and without any human interaction or intervention. In some cases, the location can be conditioned using one or more auxiliary features 247. For example, before one or more samples are taken, an auxiliary feature 247 in the form of a heater can be used to heat the surface 102 and the ground 110. When step 683 is complete, the process proceeds to optional step 684 if a sample is collected or otherwise to step 687.

In step 684, an optional step, a sample is collected. As an alternative to step 684, one or more of the sensor devices 265 can measure one or more parameters (e.g., humidity in the air 193, moisture in the ground 110) associated with a sample without collecting the sample. In certain example embodiments, the one or more samples is collected by one or more of the sample collection features 243 of the mobile testing and evaluation vehicle 101. In such a case, the one or more of the sample collection features 243 can be controlled by the control engine 406 of the controller 204 of the mobile testing and evaluation vehicle 101 using one or more algorithms 433, one or more protocols 432, and/or stored data 434 (e.g., measurements made by one or more of the sensor devices 265).

The controller 204 of the mobile testing and evaluation vehicle 101, a user 150, and/or the network manager 180 can determine how (e.g., which of the sample collection features 243 is used) the sample is collected, at what particular location in the ZOI 190 the sample is collected, and/or any other considerations relevant to the collection of the sample. In certain example embodiments, the sample is collected autonomously and without any human interaction or intervention. In some cases, the sample can be collected using one or more auxiliary features 247.

In certain example embodiments, the control engine 406 of the controller 204 of the mobile testing and evaluation vehicle 101 can physically mark the location of the mobile testing and evaluation vehicle 101 in the ZOI 190 from which the sample is collected. Examples of such markings can include, but are not limited to, stamping the surface 102 using a ZOI interaction feature 249, planting a flag in the ground 110 using an auxiliary feature 246, and spray painting the surface 102 using an auxiliary feature 246. In some cases, in the addition, or in the alternative, multiple samples can be collected and/or measured, or a long continuous sample can be collected and/or measured, while the mobile testing and evaluation vehicle 101 is moving within the ZOI 190 (as opposed to the mobile testing and evaluation vehicle 101 being stationary when one or more samples are collected and/or measured). In either case, the measurements of multiple samples can be considered individually, averaged, and/or otherwise used to evaluate the samples. When step 684 is complete, the process proceeds to step 686.

In step 686, an optional step, the sample is treated. In certain example embodiments, the one or more samples is treated by one or more of the sample treatment features 247 of the mobile testing and evaluation vehicle 101. In such a case, the one or more of the sample treatment features 247 can be controlled by the control engine 406 of the controller 204 of the mobile testing and evaluation vehicle 101 using one or more algorithms 433, one or more protocols 432, and/or stored data 434 (e.g., measurements made by one or more of the sensor devices 265).

The controller 204 of the mobile testing and evaluation vehicle 101, a user 150, and/or the network manager 180 can determine how (e.g., which of the sample treatment features 249 is used) the sample is treated, how long the sample is treated, and/or any other considerations relevant to the treatment of the sample. In certain example embodiments, the sample is treated autonomously and without any human interaction or intervention.

In step 687, one or more measurements of one or more parameters associated with a sample is obtained. As used herein, the term “obtaining” may include receiving, retrieving, accessing, generating, etc. or any other manner of obtaining the measurements. The sample can be collected prior to be measured, or the sample can be left in situ while being measured. The measurements can be obtained from one or more of the sensor devices 265. The measurements can be obtained by the control engine 406 of the controller 204 of the mobile testing and evaluation vehicle 101 using one or more algorithms 433, one or more protocols 432, and/or stored data 434. In some cases, one or more of the measurements are obtained using laboratory equipment 248. In certain example embodiments, the measurements are obtained autonomously and without any human interaction or intervention.

In step 688, the sample is evaluated based on the one or more measurements. In certain example embodiments, the sample is evaluated by the evaluation module 444 of the controller 204 of the mobile testing and evaluation vehicle 101 using one or more algorithms 433, one or more protocols 432, and/or stored data 434. The evaluation module 444 of the controller 204 of the mobile testing and evaluation vehicle 101 can determine how (e.g., whether historical measurements of the parameter are used) the sample is evaluated and/or any other considerations relevant to the evaluation of the sample. In some cases, one or more of the sample is evaluated using laboratory equipment 248. In certain example embodiments, the sample is evaluated autonomously and without any human interaction or intervention.

In certain example embodiments, the control engine 406 of the controller 204 of the mobile testing and evaluation vehicle 101 can physically mark the location of the mobile testing and evaluation vehicle 101 in the ZOI 190 from which the measurement of the sample is made. Examples of such markings can include, but are not limited to, stamping the surface 102 using a ZOI interaction feature 249, planting a flag in the ground 110 using an auxiliary feature 246, and spray painting the surface 102 using an auxiliary feature 246. In some cases, in the addition, or in the alternative, multiple measurements of multiple samples can be made by one or more of the sensor devices 265 or multiple measurements of a long continuous sample can be made by one or more of the sensor devices 265 while the mobile testing and evaluation vehicle 101 is moving within the ZOI 190 (as opposed to the mobile testing and evaluation vehicle 101 being stationary when the measurement of the sample is made).

In step 689, a determination is made as to whether one or more measurements of one or more parameters associated with another sample should be obtained. In certain example embodiments, the control engine 406 of the controller 204 of the mobile testing and evaluation vehicle 101 can make the determination as to whether measurements of parameters of another sample should be obtained. In such a case, the control engine 406 can use the evaluation module 444, the path generation module 474, one or more algorithms 433, one or more protocols 432, and/or stored data 434 (e.g., measurements made by one or more of the sensor devices 265) to make the determination. In addition, or in the alternative, the determination can be made by a user 150 communicating with the controller 204 of the mobile testing and evaluation vehicle 101. In certain example embodiments, the determination is made autonomously and without any human interaction or intervention. If one or more measurements of one or more parameters associated with another sample should be obtained, then the process proceeds to step 691. If one or more measurements of one or more parameters associated with another sample should be obtained, then the process proceeds to step 692.

In step 691, a route in the ZOI 190 is mapped to the next location. In certain example embodiments, the route in the ZOI 190 is mapped to the next location by the path generation module 474 of the controller 204 of the mobile testing and evaluation vehicle 101. In such a case, the path generation module 474 can map the route in the ZOI 190 to the next location using one or more algorithms 433, one or more protocols 432, and/or stored data 434 (e.g., measurements made by one or more of the sensor devices 265, evaluations of prior samples). The route can be unchanged from the original path or based on a modified path, both as generated by the path generation module 474.

The controller 204 (including the path generation module 474) of the mobile testing and evaluation vehicle 101, a user 150, and/or the network manager 180 can determine the path of travel to get to the next location, what objects to avoid (e.g., as determined by the object detection module 475), and/or any other considerations relevant to the mapping the route to the next location. In certain example embodiments, the route is mapped to the next location autonomously and without any human interaction or intervention. When step 691 is complete, the process reverts to step 681.

In step 692, the ZOI 190 is evaluated. In certain example embodiments, the ZOI 190 is evaluated by the evaluation module 444 of the controller 204 of the mobile testing and evaluation vehicle 101. In such a case, the evaluation module 444 can evaluate the ZOI 190 using one or more algorithms 433, one or more protocols 432, and/or stored data 434 (e.g., measurements made by one or more of the sensor devices 265, evaluations of prior samples). The controller 204 (including the evaluation module 444) of the mobile testing and evaluation vehicle 101, a user 150, and/or the network manager 180 can determine the criteria on which the ZOI 190 is evaluated and/or any other considerations relevant to the evaluation of the ZOI 190. In certain example embodiments, the ZOI 190 is evaluated autonomously and without any human interaction or intervention. When step 692 is complete, the process proceeds to the END step.

As discussed above, in some cases, multiple mobile testing and evaluation vehicles 101 are used to test and evaluate samples in adjacent ZOIs 190. In such cases, two or more of the mobile testing and evaluation vehicles 101 can communicate with each other, sharing data (e.g., measurements made by sensor devices 265, locational information, evaluations of samples). In this way, the mobile testing and evaluation vehicles 101 can coordinate with each other to evaluate one or more of the ZOIs 190.

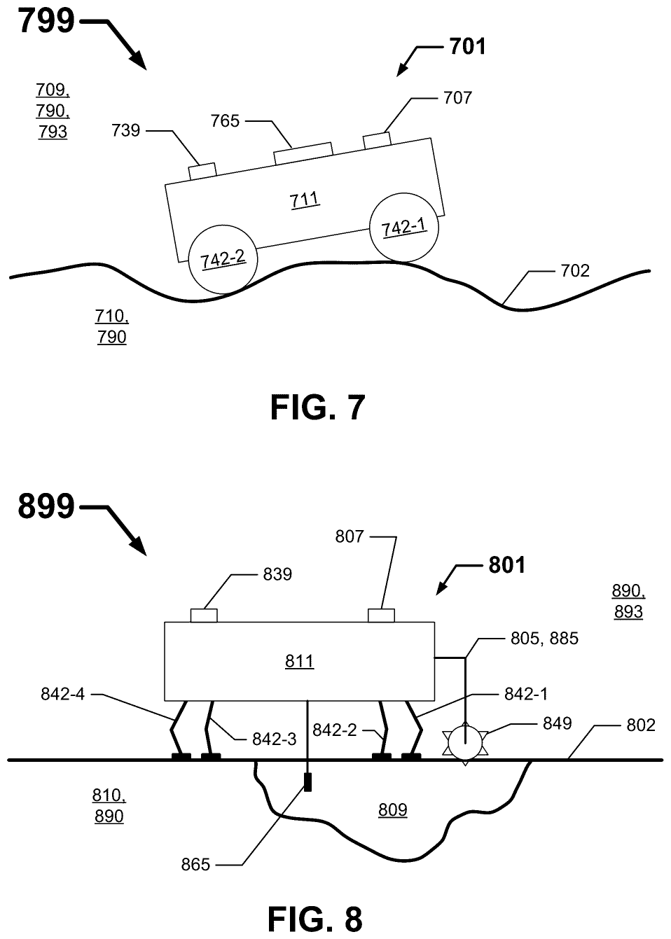

FIG. 7 shows a subsystem 799 that includes an example of a mobile testing and evaluation vehicle 701 according to certain example embodiments. Referring to FIGS. 1 through 7, the mobile testing and evaluation vehicle 701 of FIG. 7 can be substantially the same as the mobile testing and evaluation vehicle 101 discussed above with respect to FIGS. 1 through 4. For example, the mobile testing and evaluation vehicle 701 of FIG. 7 includes a body 711, a GPS 739, one or more sensor devices 765, a communication module 707, and at least two mobility features 742 (mobility feature 742-1 and mobility feature 742-2) in the form of wheels. The GPS 739, one of the sensor devices 765, and the communication module 707 are positioned atop the body 711.

The mobile testing and evaluation vehicle 701 is in a ZOI 790, which includes the air 793 and the ground 710. The COPC 709 is in the air 793 and not the ground 710. The mobility features 742 of the mobile testing and evaluation vehicle 701 make contact with the surface 702. Other components (e.g., the controller (e.g., controller 204), other sensor devices (e.g., sensor devices 265), sample collection features (e.g., sample collection features 243)) of the mobile testing and evaluation vehicle 701 are located inside the body 711 at the time captured by FIG. 7. The surface 702 in this case is undulating, which causes the mobile testing and evaluation vehicle 701 to travel in a predominately non-horizontal position.

This example highlights one of the advantages of example mobile testing and evaluation vehicles (e.g., mobile testing and evaluation vehicle 701) discussed herein. Technology used in the current art is designed for predictable or known terrains (e.g., agricultural fields, roads). Example embodiments are configured to travel over unknown vegetated or rocky terrains that may have unpredictable obstacles (e.g., large rocks, pebbles, tree roots) and unknown slopes. Specifically, the mobile testing and evaluation vehicle 701 can use on board sensor devices 765 to map out in detail the terrain within the ZOI 790, recommend the appropriate features (e.g., mobility features 242, ZOI interaction features 249, sample collection features 243) that are not already on board the mobile testing and evaluation vehicle 701, and generate/revise a path through the ZOI 790 that accounts for the terrain, including obstacles. In certain example embodiments, the evaluation module (e.g., evaluation module 444) of the controller of the mobile testing and evaluation vehicle 701 can decide whether one or more of the features on board the mobile testing and evaluation vehicle 701 should be replaced or removed based on the terrain of the ZOI 790.

In certain example embodiments, the controller of the mobile testing and evaluation vehicle 701 can use one or more sensor devices to recognize the characteristics (e.g., type of terrain, undulations) of the ZOI 790 and determine whether the mobility features 742 and/or other features (e.g., sample collection features 243, ZOI interaction features 249) are sufficient based on the characteristics of the ZOI 790. If new and/or different features are needed by the mobile testing and evaluation vehicle 701 to properly evaluate samples and evaluate the ZOI 790, the controller of the mobile testing and evaluation vehicle 701 can make that determination. In such cases, the controller of the mobile testing and evaluation vehicle 701 can notify the network manager 180 and/or a user system 155 as to the new or different features that are needed before the mobile testing and evaluation vehicle 701 can begin evaluating the samples and the ZOI 790.

In a specific application of the environment shown in FIG. 7, the mobile testing and evaluation vehicle 701 can be placed in the ZOI 790 to detect, using one or more sensor devices, mercury and/or other vapors in an effort to find the source of the gases. By using one or more other sensor devices over a relatively long (e.g., one day, several hours) period of time, the mobile testing and evaluation vehicle 701 can also measure wind speeds and direction at various locations within the ZOI 790. The controller (or portions thereof, such as the evaluation module 444) can use one or more algorithms (e.g., algorithms 433) to factor in the wind speed, wind direction, and level of mercury or other gases at various locations within the ZOI 790 to determine the source of the gases being emitted.

FIG. 8 shows a subsystem 899 that includes another example of a mobile testing and evaluation vehicle 801 according to certain example embodiments. Referring to FIGS. 1 through 8, the mobile testing and evaluation vehicle 801 of FIG. 8 can be substantially the same as the mobile testing and evaluation vehicle 101 discussed above with respect to FIGS. 1 through 4. For example, the mobile testing and evaluation vehicle 801 of FIG. 8 includes a body 811, a GPS 839, one or more sensor devices 865, a communication module 807, a ZOI interaction feature 849 in the form of a rototiller, and at least four mobility features 842 (mobility feature 842-1, mobility feature 842-2, mobility feature 842-3, and mobility feature 842-4) in the form of legs with joints and feet.

The GPS 839 and the communication module 807 are positioned atop the body 811. The sensor device 865 is inserted into the COPC 809 in the ground 810 from the bottom of the body 811 between the mobility features 842, which also extend from the bottom of the body 811. The ZOI interaction feature 849 extends from the front of the body 811 and is oriented vertically, extending below the surface 802 into the ground 810. The ZOI interaction feature 849 in this case is designed to condition the ZOI 890 (e.g., homogenize the soil) by tilling the ground 810 near the surface 802 so that the sensor device 865 can more easily be inserted into the ground 810 to take one or more subsequent measurements of the ground 810 in the COPC 809. In addition, or in the alternative, the mobility features 842 can cause the mobile testing and evaluation vehicle 801 to rotate, which has the effect of helping to homogenize the soil and/or otherwise condition the ZOI 890.

The arm between the body 811 and the ZOI interaction feature 849 can include communication links 805 and power transfer links 885 to provide power and control to the ZOI interaction feature 849. Aside from the targeted tilling of the ground 810 by the ZOI interaction feature 849, the mobility features 842 are configured to avoid substantially disturbing the ZOI 890. In some cases, some of the surface 802 and underlying ground 810 can be removed by deploying the ZOI interaction feature 849 (e.g., the rototiller) at elevated speed. During homogenization of the ZOI 890, ambient or heated air can be applied using an auxiliary feature 246 in the form of a fan or hose to reduce the moisture content of the surface 802 and/or the ground 810. In certain example embodiments, heating may be performed by the mobile testing and evaluation vehicle 801 and/or by an external device. In such cases, heating temperatures can be high enough (e.g., just over 28° F. to melt frozen seawater that contains volatile compounds, over 1000° F. to decompose a matrix) to evaluate the COPC 809 within the ZOI 890.

If the surface 802 and/or the ground 810 does not allow rototilling, the mobile testing and evaluation vehicle 801 can move in a pattern to different locations and repeat attempting to rototill until successful. The mobile testing and evaluation vehicle 801 can move a relevant sensor device (e.g., an XRF sensor, an IR sensor, a portable gas chromatograph) attached to the body 811 through a connector and moved by one or more auxiliary features 246 (e.g., in the form of motors) to the homogenized soil. In addition, or in the alternative, such a sensor device can be attached to an auxiliary feature 246 in the form of one or more arms, springs, linkages, and/or other components to position the sensor device at a desired distance from the surface 802 and/or the ground 810.

Measurements (e.g., analytical data) from the sensor device and corresponding GPS locations provided by the GPS 839 are collected and processed by the controller (including the evaluation module 444) and transmitted using the communication module 807 to a local receiver (e.g., a user system 150, the network manager 180). Such measurements can include, but are not limited to levels of H₂S, levels of Volatile Organic Carbons (VOCs), visual images and their interpretation, levels of mercury vapor, and LIDAR information obtained using the sensors devices on the mobile testing and evaluation vehicle 801. VOC monitoring allows the mobile testing and evaluation vehicle 801 to either move away from potential sources that may exceed the Lower Explosion Limit (LEL), or shut down all electricity when needed, allowing safe operations in potentially combustible environments that may be encountered at places like crude oil production sites or oil refineries. The mobile testing and evaluation vehicle 801 can collect GPS coordinates of its path from the GPS 839 and sampling locations with a time stamp (e.g., generated by the timer 435) and create an automated chain of custody.

In embodiments where the mobile testing and evaluation vehicle 801 is used to analyze the surface 802 and/or the ground 810 (e.g., soil), the mobile testing and evaluation vehicle 801 can move multiple times a short distance within the ZOI 890 to obtain additional soil surface readings of the homogenized soil. The mobile testing and evaluation vehicle 801 can move to a clean soil location and/or a cleaning station to clean the sensor device and/or the ZOI interaction feature 829 (in this case, the rototiller) to reduce cross contamination.

Advantages of using the mobile testing and evaluation vehicle 801 in the subsystem 899 of FIG. 8 can include, but are not limited to, homogenizing the samples collected and/or measured from the surface 802 and/or the ground 810, treating (e.g., drying) the samples prior to measurement and evaluation, performing measurements and evaluations in the ground 810 below the surface 802, have autonomous decision-making regarding follow-up sampling points to improve delineation with limited or no human interaction, use machine learning for continued improvement of data quality, and being able to sense combustible vapors to allow for safe deployment at sites that may have combustible vapors.

The mobile testing and evaluation vehicle 801 is in a ZOI 890, which includes the air 893 and the ground 810. The COPC 809 is in the ground 810 and not the air 893. The mobility features 842 of the mobile testing and evaluation vehicle 801 make contact with the surface 802. Other components (e.g., the controller (e.g., controller 204), other sensor devices (e.g., sensor devices 265), sample collection features (e.g., sample collection features 243)) of the mobile testing and evaluation vehicle 801 are located inside the body 811 at the time captured by FIG. 8. The surface 802 in this case is substantially planar and horizontal, which causes the mobile testing and evaluation vehicle 801 to travel in a predominately horizontal position.

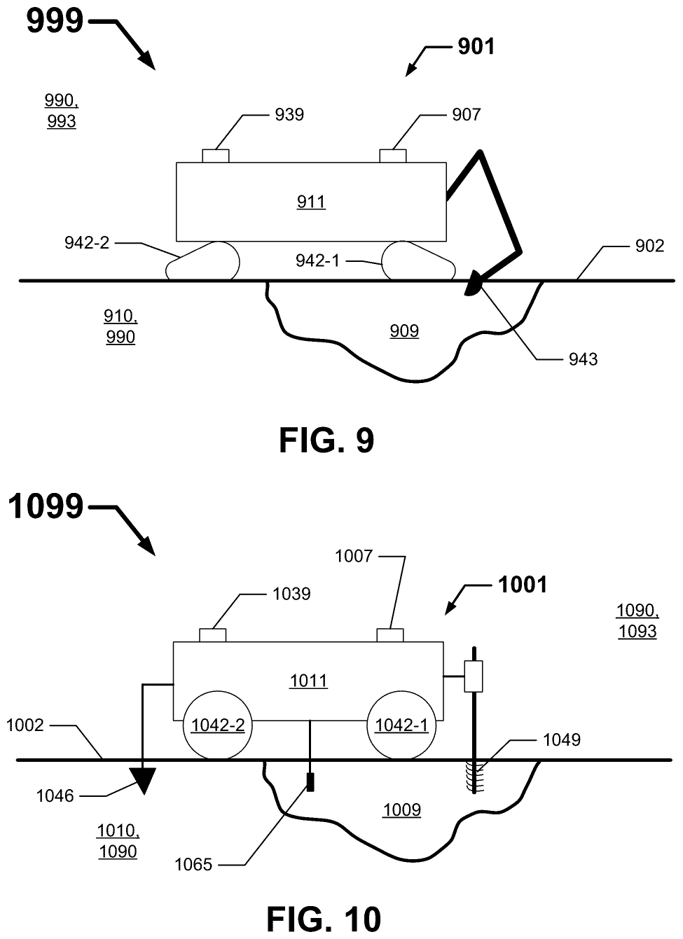

FIG. 9 shows a subsystem 999 that includes yet another example of a mobile testing and evaluation vehicle 901 according to certain example embodiments. Referring to FIGS. 1 through 9, the mobile testing and evaluation vehicle 901 of FIG. 9 can be substantially the same as the mobile testing and evaluation vehicle 101 discussed above with respect to FIGS. 1 through 4. For example, the mobile testing and evaluation vehicle 901 of FIG. 9 includes a body 911, a GPS 939, a communication module 907, a sample collection feature 943 in the form of a scooping arm or small backhoe, and at least two mobility features 942 (mobility feature 942-1 and mobility feature 942-2) in the form of caterpillar treads.

The GPS 939 and the communication module 907 are positioned atop the body 911. The sample collection feature 943 extends from the front of the body 911 with an arm that is hinged along several locations and has the scoop at its far end so that the scoop can extend below the surface 902 into the ground 910. The sample collection feature 943 in this case is designed to collect samples from the ground 910 in the scoop. In this case, the sample is also in the COPC 909. Once the sample is collected by the sample collection feature 943, a sensor device (e.g., sensor device 265) located inside the body 911 can measure one or more parameters associated with the sample.

The mobile testing and evaluation vehicle 901 is in a ZOI 990, which includes the air 993 and the ground 910. The COPC 909 is in the ground 910 and not the air 993. The mobility features 942 of the mobile testing and evaluation vehicle 901 make contact with the surface 902. Other components (e.g., the controller (e.g., controller 204), the sensor devices (e.g., sensor devices 265)) of the mobile testing and evaluation vehicle 901 are located inside the body 911 at the time captured by FIG. 9. The surface 902 in this case is substantially planar and horizontal, which causes the mobile testing and evaluation vehicle 901 to travel in a predominately horizontal position.

FIG. 10 shows a subsystem 1099 that includes still another example of a mobile testing and evaluation vehicle 1001 according to certain example embodiments. Referring to FIGS. 1 through 10, the mobile testing and evaluation vehicle 1001 of FIG. 10 can be substantially the same as the mobile testing and evaluation vehicle 101 discussed above with respect to FIGS. 1 through 4. For example, the mobile testing and evaluation vehicle 1001 of FIG. 10 includes a body 1011, a GPS 1039, one or more sensor devices 1065, a communication module 1007, a ZOI interaction feature 1049 in the form of an auger, and at least two mobility features 1042 (mobility feature 1042-1 and mobility feature 1042-2) in the form of wheels.

The GPS 1039 and the communication module 1007 are positioned atop the body 1011. The sensor device 1065 is inserted into the COPC 1009 in the ground 1010 from the bottom of the body 1011 between the mobility features 1042. The ZOI interaction feature 1049 extends from the front of the body 1011 and is oriented vertically, extending below the surface 1002 into the ground 1010. The ZOI interaction feature 1049 in this case is designed to condition the ZOI 1090 by creating a hole in the ground 1010 near the surface 1002 so that the sensor device 1065 can more easily be inserted into the ground 1010 to take one or more subsequent measurements of the ground 1010 in the COPC 1009.

Since the mobile testing and evaluation vehicle 1001 may not weigh enough to allow for the ZOI interaction feature 1049 to be properly leveraged, an auxiliary feature 1046 in the form of an anchor embedded in the ground 1010 is positioned at the rear end (e.g., opposite the front end where the ZOI interaction feature 1049) of the body 1011. In this case, the auxiliary feature 1046 can be hammered into the ground 1010 by another auxiliary feature in the form of a hammer. As another example, the auxiliary feature 1046 can be in the form of a spiral soil anchor that is screwed into the ground 1010 by another auxiliary feature in the form of a drill or similar rotational element that creates torque on the auxiliary feature 1046. In any case, the same or a different auxiliary feature can be used to remove the auxiliary feature 1046 from the ground 1010 when the mobile testing and evaluation vehicle 1001 is ready to be moved to another location within the ZOI 1090.

The mobile testing and evaluation vehicle 1001 is in a ZOI 1090, which includes the air 1093 and the ground 1010. The COPC 1009 is in the ground 1010 and not the air 1093. The mobility features 1042 of the mobile testing and evaluation vehicle 1001 make contact with the surface 1002. Other components (e.g., the controller (e.g., controller 204), other sensor devices (e.g., sensor devices 265)) of the mobile testing and evaluation vehicle 1001 are located inside the body 1011 at the time captured by FIG. 10. The surface 1002 in this case is substantially planar and horizontal, which causes the mobile testing and evaluation vehicle 1001 to travel in a predominately horizontal position.

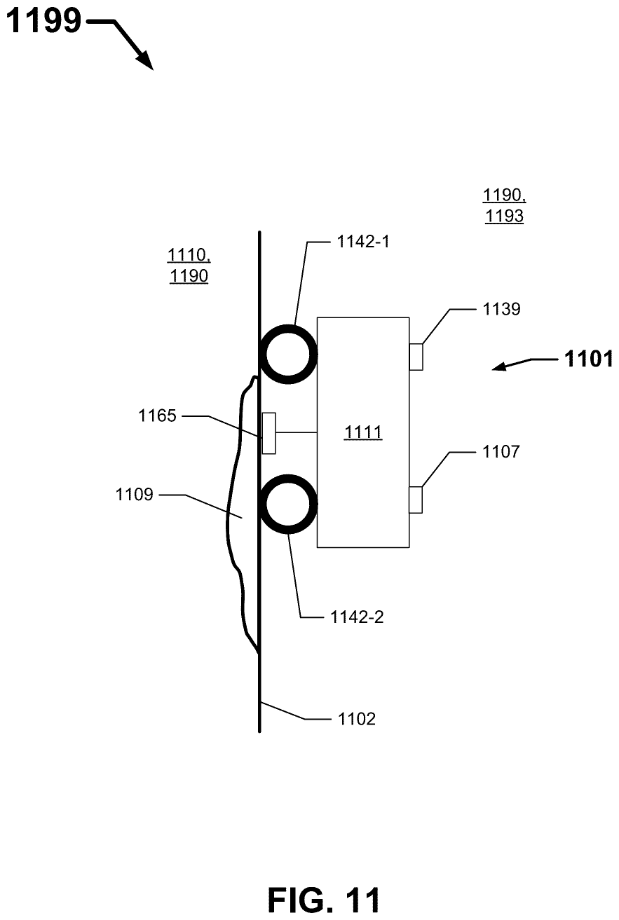

FIG. 11 shows a subsystem 1199 that includes yet another example of a mobile testing and evaluation vehicle 1101 according to certain example embodiments. Referring to FIGS. 1 through 11, the mobile testing and evaluation vehicle 1101 of FIG. 11 can be substantially the same as the mobile testing and evaluation vehicle 101 discussed above with respect to FIGS. 1 through 4. For example, the mobile testing and evaluation vehicle 1101 of FIG. 11 includes a body 1111, a GPS 1139, one or more sensor devices 1165, a communication module 1107, and at least two mobility features 1142 (mobility feature 1142-1 and mobility feature 1142-2) in the form of magnetic wheels.

The GPS 1139 and the communication module 1107 are positioned atop the body 1111. The sensor device 1165 is located below the bottom of the body 1111 between the mobility features 1142 and is adjacent to the surface 1102. The surface 1102, part of the side wall of a tank, is planar and vertically oriented. The COPC 1109 is in part of the thickness of the wall 1110 (the equivalent of the ground 110 of FIG. 1). The mobile testing and evaluation vehicle 1101 is in a ZOI 1190, which includes the air 1193 and the wall 1110 of the tank. The COPC 1109 is in part of the wall 1110 and not the air 1193. The mobility features 1142 of the mobile testing and evaluation vehicle 1101 make contact with the surface 1102. Because the mobility features are magnetized, the mobile testing and evaluation vehicle 1101 does not fall despite being vertically oriented. Other components (e.g., the controller (e.g., controller 204), other sensor devices (e.g., sensor devices 265)) of the mobile testing and evaluation vehicle 1101 are located inside the body 1111 at the time captured by FIG. 11. The surface 1102 in this case is substantially planar and vertical, which causes the mobile testing and evaluation vehicle 1101 to travel in a predominately vertical position.

This example embodiment shows that the mobile testing and evaluation vehicle 1101 can be used with surface-impacted bodies (e.g., steel bodies, metallic bodies), such as vessels, tanks, ship holds, and pipelines. Example embodiments of the mobile testing and evaluation vehicle 1101 may include a site-specific sensor device 1165 (e.g., a detector) mounted on the body 1011 that uses mobility features 1142 in the form of magnets or other features (e.g., dry/directional adhesive, suction cups) to connect to the surface 1102 of the site via magnetism, van der Waals interactions, and/or types of interactions. In this example, where the mobility features 1142 are in the form of magnetic wheels, the mobile testing and evaluation vehicle 1101 can move on steel slopes, vertical steel walls, and/or in upside down positions. A mobile testing and evaluation vehicle 1101 with such a configuration can move to specific pre-programmed locations (according to the controller or portions thereof) and collect measurements on the surface 1102. The measurements can be used to create a heat map of the distribution of the COPC 1109 on the surface 1102 of the site in real time, with an algorithm (e.g., algorithm 433) being used to determine where to collect the next sample.

By way of non-limiting example, the matrix of the surface 1102 may include a polymer coating, corrosion material, or scale deposits. The mobile testing and evaluation vehicle 1101 can move a specific sensor device 1165 (e.g., a NPK detector, a moisture detector, a CO₂ sensor, a carbon isotope detector, an XRF detector, an IR detector, a radiation detector) to the surface 1102 and collect a measurement. An evaluation of these measurements can be used to assess bioremediation potential. As an example, the mobile testing and evaluation vehicle 1101 may include an auxiliary feature (e.g., auxiliary feature 246) in the form of an arm or similar feature that is capable of moving one or more sensor devices 1165 to various locations such as a site floor, wall, ceiling, and other features such as ladders, trusses, and the like. The sensor devices 1165 may be integrated onto one or more of the auxiliary features individually or in combination (e.g., the mobile testing and evaluation vehicle 1101 may include one or more auxiliary features, each having one or more sensor devices).

In certain example embodiments, a ZOI interaction feature (e.g., ZOI interaction feature 249) in the form of a surface agitator (e.g., a grinder, a sander, a drill), such as what is shown above in FIG. 8, may be used prior to an analysis to expose materials under the surface 1102, including the unimpacted base metal. In such cases, a sensor device 1165 in the form of a gas vapor monitor can measure the potential presence of combustible gases to ensure safe operation and allow retraction or shutdown of the mobile testing and evaluation vehicle 1101. The mobile testing and evaluation vehicle 1101 may also be configured to monitor the presence of mercury vapor. For example, the mobile testing and evaluation vehicle 1101 may use a sensor device 115 in the form of an on-board XRF and/or a portable mercury vapor sensor to evaluate potential risks for entry and sources of mercury.

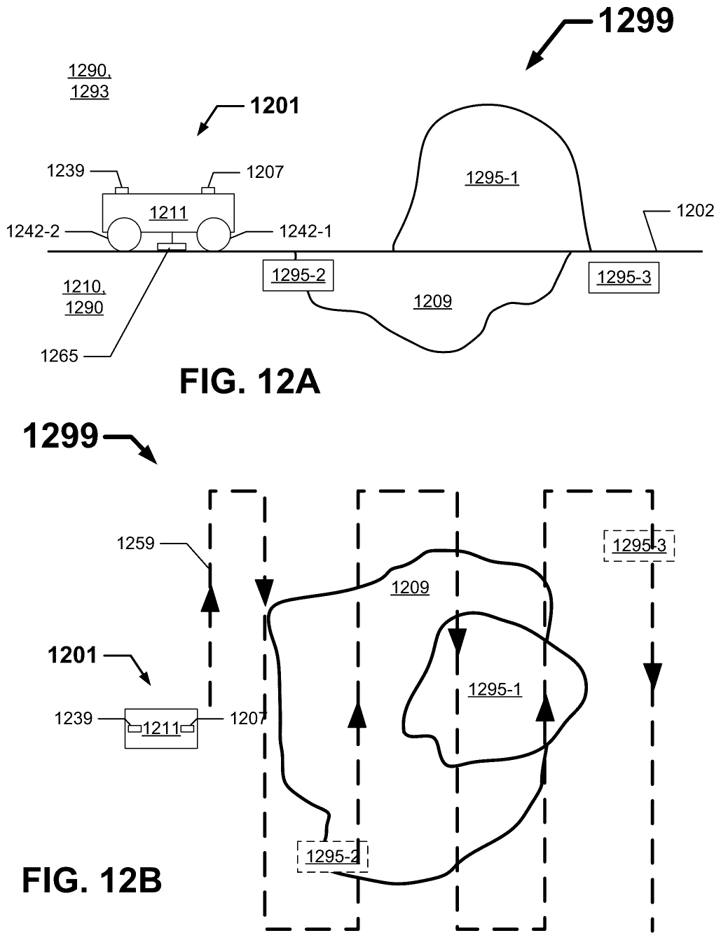

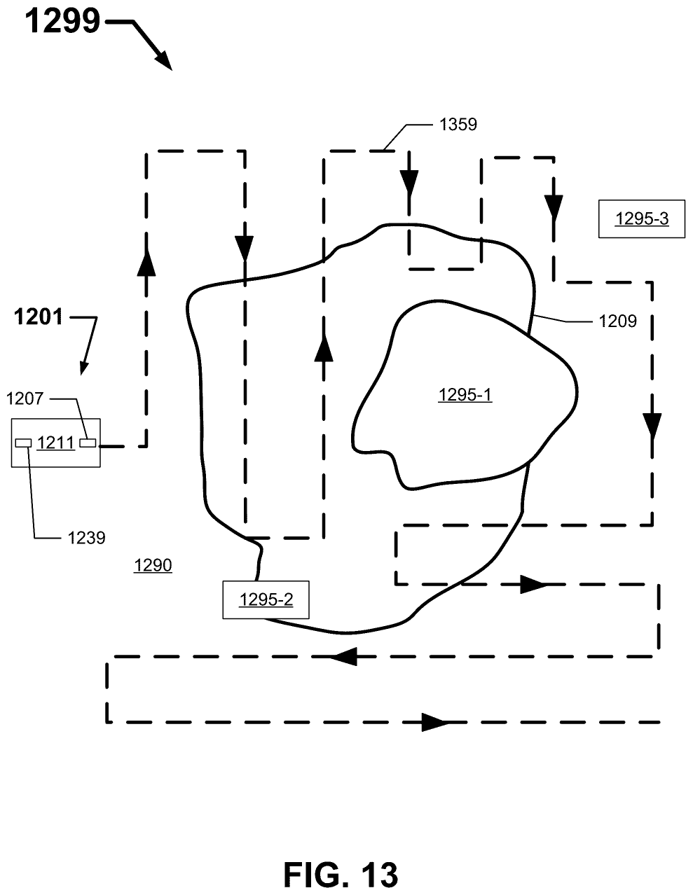

FIGS. 12A and 12B show a subsystem 1299 that includes an example of a mobile testing and evaluation vehicle 1201 identifying objects 1295 in a zone of interest according to certain example embodiments. Specifically, FIG. 12A shows a side view of the subsystem 1299, and FIG. 12B shows a top view of the subsystem 1299. FIG. 13 shows diagram of the subsystem 1299 of FIGS. 12A and 12B with a path 1359 generated by the example mobile testing and evaluation vehicle based on identifying the objects according to certain example embodiments. Referring to FIGS. 1 through 13, the mobile testing and evaluation vehicle 1201 of FIGS. 12A through 13 can be substantially the same as the mobile testing and evaluation vehicle 101 discussed above with respect to FIGS. 1 through 4. For example, the mobile testing and evaluation vehicle 1201 of FIGS. 12A through 13 includes a body 1211, a GPS 1239, one or more sensor devices 1265, a communication module 1207, and at least two mobility features 1242 (mobility feature 1242-1 and mobility feature 1242-2) in the form of wheels.

The GPS 1239 and the communication module 1207 are positioned atop the body 1211. The sensor device 1265 is located below the bottom of the body 1211 between the mobility features 1242 and is adjacent to the surface 1202. The surface 1202 is planar and horizontally oriented. The COPC 1209 is in the ground 1210 and not the air 1293. The mobility features 1242 of the mobile testing and evaluation vehicle 1201 make contact with the surface 1202. Other components (e.g., the controller (e.g., controller 204), other sensor devices (e.g., sensor devices 265)) of the mobile testing and evaluation vehicle 1201 are located inside the body 1211 at the time captured by FIGS. 12A through 13. The surface 1202 in this case is substantially planar and horizontal, which causes the mobile testing and evaluation vehicle 1201 to travel in a predominately horizontal position.

There are three objects 1295 in the subsystem 1299. Object 1295-1 is in the form of a huge boulder or pile of rocks that sit atop the surface 1202 and rise into the air 1293. In alternative embodiments, object 1295-1 can be another type of obstacle in the ZOI 1290 that should be avoided by the mobile testing and evaluation vehicle 1201. Examples of such other forms of the object 1295-1 can include, but are not limited to, an environmentally protected area, a storage tank, an area with rare or sensitive vegetation, and an area (e.g., deep mud) that can cause the mobile testing and evaluation vehicle 1201 to become stuck. Object 1295-2 and object 1295-3 are each in the form of land mines or improvised explosive devices (IEDs) buried in the ground 1210 close to the surface 1202.

The objects 1295 can be located and/or identified by the mobile testing and evaluation vehicle 1201 using measurements made by one or more of the sensor devices (e.g., sensor devices 265) and the controller (e.g., controller 204). For example, if a sensor device of the mobile testing and evaluation vehicle 1201 is a camera, the controller of the mobile testing and evaluation vehicle 1201 can interpret and characterize (e.g., in terms of shape, in terms of size, in terms of location, in terms of form) the object 1295-1 in the images captured by the camera. As another example, if two sensor devices of the mobile testing and evaluation vehicle 1201 are ground penetrating radar and a metal detector, the controller of the mobile testing and evaluation vehicle 1201 can use the information provided by those sensor devices to interpret and identify (e.g., in terms of shape, in terms of size, in terms of location, in terms of form) the object 1295-2 and the object 1295-3. In alternative embodiments, the controller of the mobile testing and evaluation vehicle 1201 can communicate with the network manager 180 and/or a user system 155 to establish the characteristics of the objects 1295.

In this case, all of these objects 1295 need to be avoided by the mobile testing and evaluation vehicle 1201 as it moves to evaluate the samples and the ZOI 1290. Specifically, the mobility features 1242 of the mobile testing and evaluation vehicle 1201 in this case are not equipped to climb the object 1295-1, and so the object 1295-1 must be avoided by the mobile testing and evaluation vehicle 1201. Also, if the mobile testing and evaluation vehicle 1201 get too close to object 1295-2 or object 1295-3, a detonation could occur that would destroy or severely damage the mobile testing and evaluation vehicle 1201. Most of the object 1295-1 is located above the COSC 1209, most of the object 1295-2 is located in the COSC 1209, and none of the object 1295-2 is located in the COSC 1209-3.

With this information, the path generation module (e.g., path generation module 474) of the controller of the mobile testing and evaluation vehicle 1201 can generate a path 1359 by which the mobile testing and evaluation vehicle 1201 can travel to evaluate samples and evaluate the ZOI 1290 (and, more specifically, the COSC 1209 therein). As shown in FIG. 13, the path 1359 avoids all of the objects 1295 to avoid damage to and/or destruction of the mobile testing and evaluation vehicle 1201. In some cases, the path 1359 can be an initial path generated by the path generation module. Alternatively, the path 1359 can be modified by the path generation module from an initial path 1259 that was established before the objects 1295 were identified and that ran through the space occupied by the object 1295-1 and/or ran over the object 1295-2 and/or the object 1295-3. The initial path 1259, shown in FIG. 12B, runs over all three objects 1295.

In some cases, depending on the sample collection features (e.g., sample collection features 243) on board the mobile testing and evaluation vehicle 1201, one or more samples can be collected underneath the object 1295-1 when the mobile testing and evaluation vehicle 1201 is located adjacent to the object 1295-1 along the revised path 1359. For example, if a sample collection feature of the mobile testing and evaluation vehicle 1201 is a core plug that can be extended downward and outward from the mobile testing and evaluation vehicle 1201, then one or more samples can be collected in the COPC 1209 under the object 1295-1.

Some advantages of using example mobile testing and evaluation vehicles (e.g., mobile testing and evaluation vehicle 1201) can include, but are not limited to, little or no disturbance of the surface 1202 and/or the ground 1210 (e.g., no soil disturbance), no human handling of the COPC 1209 (e.g., no soil handling), little or no waste, little or no cross-contamination of samples, and acquisition of real time data (e.g., measurements) that can be used to make field decisions (e.g., made by the controller of the mobile testing and evaluation vehicle 1201, made by a user 150, made by the network manager 180) such as selection of follow-up sampling locations within the ZOI 1290.

Based on site-specific criteria, the mobile testing and evaluation vehicle 1201 may use a uniform algorithm (e.g., algorithm 433) such as the Boustrophedon path sample planner to perform an initial site screening, set up pre-programmed sampling locations, and/or use existing site data to obtain a first screening of the ZOI 1290 to create a “heat map”, which may indicate the relative intensity of location-correlated impacts (e.g., localized contamination concentrations) of the COPC 1209.

The mobile testing and evaluation vehicle 1201 can then use the field data and an optimization algorithm (e.g., algorithm 433) to determine locations for follow-up sampling within the ZOI 1290. Such a process can have one or more benefits, such as improving contaminant delineation or identifying hot-spots (e.g., areas with progressively increasing contaminant concentrations) to improve the quality (e.g., accuracy and precision) of the heat map. The mobile testing and evaluation vehicle 1201 can continue to use new data to determine the location of follow-up samples until the desired data quality or sampling time frame has been reached.

As a specific example, in certain example embodiments, a sensor device 1265 in the form of a Lidar detector in combination with a camera and obstacle recognition software (used by the controller) can be used to detect and circumvent objects 1295-1 that may be too big to be cleared by the mobile testing and evaluation vehicle 1201. The path generation module of the controller can build a map of the ZOI 1290 and localize readings within that map. The mobile testing and evaluation vehicle 1201 can be configured to move along the revised path 1359 autonomously or through remote operation. Measurements obtained by the controller of the mobile testing and evaluation vehicle 1201, including surface, gas, and visual measurements, can be stored and processed by the controller (including portions thereof) and/or transmitted to a remote receptor using the communication module 1207. Distances moved by the mobile testing and evaluation vehicle 1201 can be tracked by using the GPS 1239 and/or by measuring wheel rotations (or other measurement of movement cycles) and direction to track the location of the mobile testing and evaluation vehicle 1201, as well as using the LIDAR, cameras, and/or other sensor devices for localization. One or more algorithms can be used to select follow-up sampling locations based on data obtained by the mobile testing and evaluation vehicle 1201.

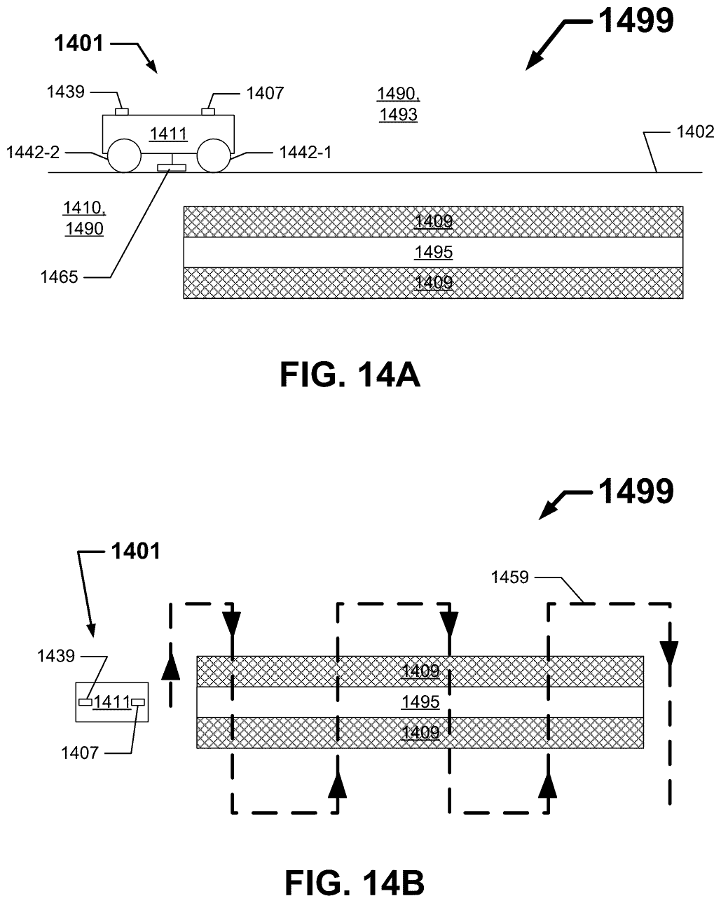

FIGS. 14A and 14B show a subsystem 1499 that includes an example mobile testing and evaluation vehicle 1401 generating a path 1459 based on identifying an object 1495 in a ZOI 1490 according to certain example embodiments. Specifically, FIG. 14A shows a side view of the subsystem 1499 before the path 1459 is generated, and FIG. 14B shows a top view of the subsystem 1499 after the path 1459 is generated. Referring to FIGS. 1 through 14B, the mobile testing and evaluation vehicle 1401 of FIGS. 14A and 14B can be substantially the same as the mobile testing and evaluation vehicle 101 discussed above with respect to FIGS. 1 through 4. For example, the mobile testing and evaluation vehicle 1401 of FIGS. 14A and 14B includes a body 1411, a GPS 1439, one or more sensor devices 1465, a communication module 1407, and at least two mobility features 1442 (mobility feature 1442-1 and mobility feature 1442-2) in the form of wheels.

The GPS 1439 and the communication module 1407 are positioned atop the body 1411. The sensor device 1465 is located below the bottom of the body 1411 between the mobility features 1442 and is adjacent to the surface 1402. The surface 1402 is planar and horizontally oriented. The COPC 1409 is in the ground 1410 surrounding an object 1495 in the form of a buried pipe. The COPC 1409 is not in the air 1493. The mobility features 1442 of the mobile testing and evaluation vehicle 1401 make contact with the surface 1402. Other components (e.g., the controller (e.g., controller 204), other sensor devices (e.g., sensor devices 265)) of the mobile testing and evaluation vehicle 1401 are located inside the body 1411 at the time captured by FIGS. 14A and 14B. The surface 1402 in this case is substantially planar and horizontal, which causes the mobile testing and evaluation vehicle 1401 to travel in a predominately horizontal position.

In this case, the object 1495 is being sought rather than avoided. Specifically, the mobile testing and evaluation vehicle 1401 in this example is being deployed to collect and/or evaluate samples surrounding the object 1495, and then evaluate the ZOI 1490 (and, more specifically, the COPC 1409) based on the evaluation of the samples. To accomplish its task, the mobile testing and evaluation vehicle 1401 must first locate the object 1495, and then generate a path 1459 based on the location of the object 1495. The object 1495 can be located and/or identified by the mobile testing and evaluation vehicle 1401 using measurements made by the sensor device 1465 (e.g., in the form of ground penetrating radar) and/or one or more of the other sensor devices (e.g., sensor devices 265) of the mobile testing and evaluation vehicle 1401. With these measurements, the controller (e.g., controller 204) of the mobile testing and evaluation vehicle 1401 can identify (e.g., characterize) and locate the object 1495. In alternative embodiments, the controller of the mobile testing and evaluation vehicle 1401 can communicate with the network manager 180 and/or a user system 155 to establish the characteristics of the object 1495.

With this information, the path generation module (e.g., path generation module 474) of the controller of the mobile testing and evaluation vehicle 1401 can generate a path 1459 by which the mobile testing and evaluation vehicle 1401 can travel to evaluate samples and evaluate the ZOI 1490 (and, more specifically, the COSC 1409 therein) proximate to the object 1495. As shown in FIG. 14B, the path 1459 covers areas over and proximate to the object 1495. In some cases, the path 1459 can be an initial path generated by the path generation module. Alternatively, the path 1459 can be modified by the path generation module from some other initial path that was established before the object 1495 was identified and located.

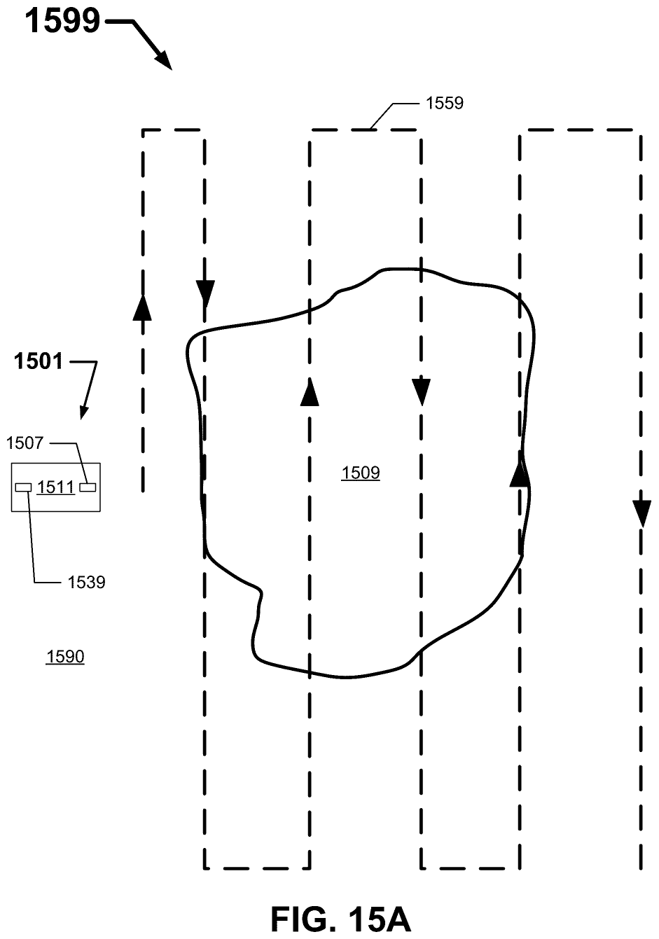

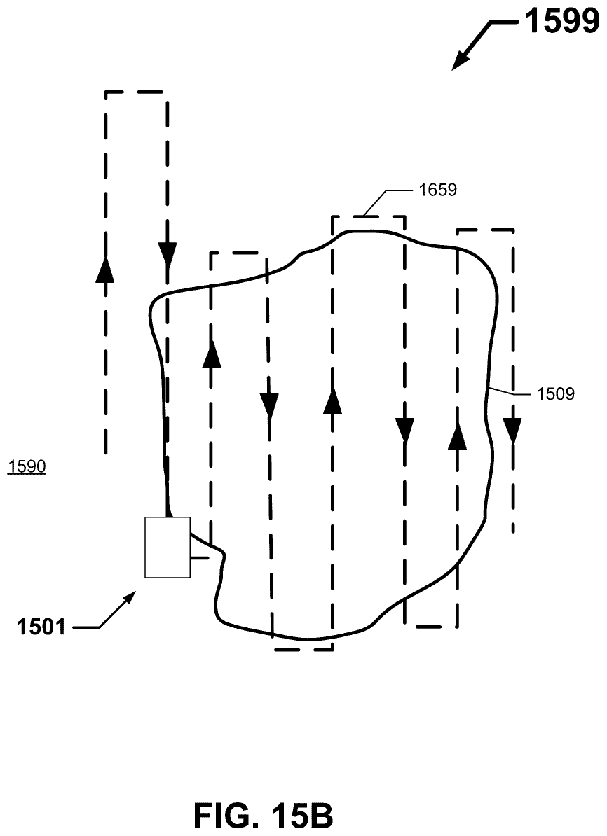

FIGS. 15A and 15B show a subsystem 1599 that includes an example mobile testing and evaluation vehicle 1501 modifying a path based on evaluating samples in a ZOI 1590 according to certain example embodiments. Specifically, FIG. 15A shows a top view of the subsystem 1599 with the initial path 1559, and FIG. 15B shows a top view of the subsystem 1599 with the modified path 1659. Referring to FIGS. 1 through 15B, the mobile testing and evaluation vehicle 1501 of FIGS. 15A and 15B can be substantially the same as the mobile testing and evaluation vehicle 101 discussed above with respect to FIGS. 1 through 4. For example, the mobile testing and evaluation vehicle 1501 of FIGS. 15A and 15B includes a body 1511, a GPS 1539, and a communication module 1507. The mobility features are hidden from view by the body 1511.

The GPS 1539 and the communication module 1507 are positioned atop the body 1511. The COPC 1509 can be in the ground (e.g., ground 110), on the surface (e.g., surface 102), and/or in the air (e.g., air 193). Other components (e.g., the controller (e.g., controller 204), other sensor devices (e.g., sensor devices 265)) of the mobile testing and evaluation vehicle 1501 are located inside the body 1511 at the times captured by FIGS. 15A and 15B. The mobile testing and evaluation vehicle 1501 begins traveling the original path 1559 shown in FIG. 15A. At the time shown in FIG. 15B, based on evaluation of the samples from the COPC 1509 that are traversed by the original path 1559, followed by the evaluation of samples that are outside of the COPC 1509, the path generation module (e.g., path generation module 474) of the controller of the mobile testing and evaluation vehicle 1501 determines that a revised path 1659 should be generated so that resources are not wasted on evaluating too many samples outside the COPC 1509.

The revised path 1659 can in turn be revised, based on subsequent evaluations of samples, one or more times by the path generation module before the mobile testing and evaluation vehicle 1501 is done evaluating the ZOI 1590. This revised path 1659 can be generated based on adaptive sampling, where the mobile testing and evaluation vehicle 1501 generates the revised path 1659 in an effort to find other samples from the COPC 1509. The path generation module of the controller of the mobile testing and evaluation vehicle 1501 can use one or more algorithms (e.g., algorithms 433) to generate the revised path 1659. Such algorithms can be or include a stastical (e.g., Gaussian) reasoning function.

Example embodiments can be used to evaluate one or more samples proximate to and/or within a COPC in a ZOI. Such evaluations can be made using measurements of parameters from one or more sensor devices on board an example mobile testing and evaluation vehicle. An example mobile testing and evaluation vehicle can work in conjunction with one or more other mobile testing and evaluation vehicles. Example mobile testing and evaluation vehicles can identify objects to be avoided and modify paths of travel. The various features of example mobile testing and evaluation vehicles can be replaced, added, removed, and/or changed so that the features are suitable for the particular terrain encountered in the ZOI. Example embodiments eliminate the need for human involvement in the collection of samples, the evaluation of samples, and the evaluation of a COPC within a ZOI. Example embodiments also provide a number of other benefits. Such other benefits can include, but are not limited to, less use of resources, greater operational flexibility, time savings, improved personnel safety, and compliance with applicable industry standards and regulations.

Although embodiments described herein are made with reference to example embodiments, it should be appreciated by those skilled in the art that various modifications are well within the scope and spirit of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

What is claimed is:

1. A mobile testing and evaluation vehicle comprising:
a body;
a mobility feature disposed on the body, wherein the mobility feature is configured to move the body with respect to a zone of interest;
a sensor device configured to obtain a measurement of a parameter associated with a sample in the zone of interest; and
a controller configured to:
receive the measurement;
evaluate the sample based on the measurement; and
evaluate the zone of interest based on evaluating the sample.

2. The mobile testing and evaluation vehicle of claim 1, wherein the body is intrinsically safe.

3. The mobile testing and evaluation vehicle of claim 1, wherein the mobility feature is configured to move the body in a substantially vertical plane.

4. The mobile testing and evaluation vehicle of claim 1, further comprising:
a sample collection feature coupled to the body, wherein the sample collection feature is configured to collect the sample from the zone of interest.

5. The mobile testing and evaluation vehicle of claim 4, further comprising:
a sample treatment feature configured to treat the sample before the measurement of the parameter associated with the sample is obtained.

6. The mobile testing and evaluation vehicle of claim 1, further comprising:
a zone of interest interaction feature coupled to the body, wherein the zone of interest interaction feature is configured to manipulate the zone of interest.

7. The mobile testing and evaluation vehicle of claim 1, wherein the mobility feature is configured to move the body without substantially disturbing the zone of interest.

8. The mobile testing and evaluation vehicle of claim 1, wherein evaluating the sample by the controller comprises executing an algorithm using a value associated with the measurement.

9. The mobile testing and evaluation vehicle of claim 1, wherein the zone of interest is under water, and wherein the body is configured to be water-proof.

10. The mobile testing and evaluation vehicle of claim 1, wherein the sensor device is further configured to identify an object to avoid within the zone of interest.

11. A method for assessing a site with compounds of potential concern, the method comprising:
moving, by a mobile testing and evaluation vehicle, to a plurality of locations in a zone of interest;
obtaining, by a mobile testing and evaluation vehicle, a plurality of measurements of a parameter associated with a plurality of samples in the zone of interest;
evaluating, by the mobile testing and evaluation vehicle, the plurality of samples based on the plurality of measurements; and
evaluating, by the mobile testing and evaluation vehicle, the zone of interest based on evaluating the plurality of samples.

12. The method of claim 11, further comprising:
determining, by the mobile testing and evaluation vehicle, a path along which that the plurality of measurements are obtained.

13. The method of claim 12, further comprising:
modifying, by the mobile testing and evaluation vehicle, the path based on evaluating some of the plurality of samples.

14. The method of claim 12, further comprising:
identifying, by the mobile testing and evaluation vehicle, an object to avoid, wherein the path is modified to avoid the object.

15. The method of claim 11, further comprising:
conditioning, by the mobile testing and evaluation vehicle, part of the zone of interest.

16. The method of claim 11, further comprising:
collecting, by the mobile testing and evaluation vehicle, the plurality of samples from the zone of interest prior to obtaining the plurality of measurements of the parameter associated with the plurality of samples.

17. The method of claim 16, further comprising:
treating, by the mobile testing and evaluation vehicle, the plurality of samples prior to obtaining the plurality of measurements of the parameter associated with the plurality of samples.

18. The method of claim 16, further comprising:
marking, by the mobile testing and evaluation vehicle, a location where each of the plurality of samples is collected.

19. The method of claim 11, further comprising:
coordinating, by the mobile testing and evaluation vehicle, with an additional mobile testing and evaluation vehicle to evaluate the zone of interest.

20. The method of claim 11, wherein the plurality of measurements is obtained while the mobile testing and evaluation vehicle is moving within the zone of interest.
